(19) United States
(12) Patent Application Publication
(10) Pub. No.: US 2025/0130230 A1
(43) Pub. Date: Apr. 24, 2025

(54) METHODS, DEVICES, AND RELATED ASPECTS FOR DETECTING SEVERE ACUTE RESPIRATORY SYNDROME CORONAVIRUS-2

(71) Applicants: ARIZONA BOARD OF REGENTS ON BEHALF OF ARIZONA STATE UNIVERSITY, Scottsdale, AZ (US); UNIVERSITY OF WASHINGTON, SEATTLE, WA (US)

(72) Inventors: Chao Wang, Chandler, AZ (US); Liangcai Gu, Seattle, WA (US); Xiahui Chen, Mesa, AZ (US); Shoukai Kang, Seattle, WA (US); MD Ashif Ikbal, Tempe, AZ (US); Zhi Zhao, Mesa, AZ (US)

(73) Assignees: ARIZONA BOARD OF REGENTS ON BEHALF OF ARIZONA STATE UNIVERSITY, Scottsdale, AZ (US); UNIVERSITY OF WASHINGTON, SEATTLE, WA (US)

(21) Appl. No.: 18/691,615

(22) PCT Filed: Sep. 27, 2022
(86) PCT No.: PCT/US22/77110

§ 371 Date: Mar. 13, 2024

Related U.S. Application Data
(60) Provisional application No. 63/250,023, filed on Sep. 29, 2021.

(51) Int. Cl. G01N 33/569; G01N 33/543; G01N 33/553

(52) U.S. Cl. CPC G01N 33/56983; G01N 33/54346; G01N 33/54386; G01N 33/553; G01N 2333/165; G01N 2469/10; G01N 2470/04

(57) ABSTRACT

Provided herein are methods of detecting severe acute respiratory syndrome coronavirus-2 (SARS-CoV-2) in a sample. The methods include contacting the sample with a plurality of gold nanoparticles (AuNPs) and/or other plasmonic metal nanoparticles (MNPs) that are conjugated with at least two sets of antibodies, or antigen binding portions thereof, that binds to at least first and second epitopes of SARS-CoV-2 proteins, such as receptor-binding domain (RBDs) under conditions sufficient for the antibodies, or the antigen binding portions thereof, to bind to the first and second epitopes of the SARS-CoV-2 proteins in the sample to produce bound SARS-CoV-2 proteins. The methods also include detecting the SARS-CoV-2 proteins when aggregations of the bound SARS-CoV-2 proteins form with one another. Related compositions, reaction mixtures, devices, kits, and systems are also provided.

### CROSS-REFERENCE TO RELATED APPLICATIONS

This application claims priority to U.S. Provisional Patent Application Ser. No. 63/250,023, filed Sep. 29, 2021, the disclosure of which is incorporated herein by reference.

### STATEMENT REGARDING FEDERALLY SPONSORED RESEARCH OR DEVELOPMENT

This invention was made with government support under 1809997, 1847324, 2020464, and 1542160 awarded by the National Science Foundation and R35 GM128918 awarded by the National Institutes of Health. The government has certain rights in the invention.

### BACKGROUND

The coronavirus disease (COVID-19), caused by the RNA virus severe acute respiratory syndrome coronavirus 2 (SARS-CoV-2), has infected >22 million people and caused >780,000 deaths in >210 countries, states, or territories, with >250,000 new cases daily. There are already >5.6 million cases and >175,000 deaths in the U.S., and these statistics continue to worsen. Given the high mortality rate (1-3% compared to 0.1% for influenza), fast transmission (reproductive number R0 is estimated 3-4 but significantly higher in densely populated areas), asymptotic infection in some individuals, relatively long hospitalization (˜2 weeks in average), and unavailable effective drugs or vaccines, COVID-19 has posed an unprecedented threat to human health and economics in the U.S. and the world.

Currently, most COVID-19 diagnostics are based on reverse-transcription polymerase chain reaction (RT-PCR) assay, enzyme-linked immunosorbent assay (ELISA) and colloidal gold or fluorescence based rapid immunoassay. RT-PCR and ELISA generally require skilled medical practitioners operating bulky equipment and following elaborate diagnostics protocols that can takes up to 5 hours to complete. In addition, centralized laboratories with well-established COVID-19 diagnostics capacities are generally greatly limited due to the strict biosafety regulations (BSL-3), which significantly reduce the diagnostics throughput and increase diagnostic cost taking into account the biological sample shipment charges from the epidemic region to the laboratories. These methods, although widely used in clinical diagnostics, generally lack the stringent requirements of quick, user-friendly, electricity-free and low cost in field point-of-care (POC) solutions to COVID-19 diagnostics in medical resource limited regions.

Rapid lateral flow immunochromatographic technology has been widely adopted in POC applications. It generally meets POC requirements such as fast screening speed, low cost and zero electricity consumption. However, its detection sensitivity is relatively low (up to hundreds of ng/ml in antigen protein detection) compared to RT-PCR and ELISA and entails qualitative analysis unless coupled to readout devices. In addition, there have been confirmed false positive cases (as high as 20%) from field validation, which can leave patients exposed to nosocomial SARS-CoV-2 transmission, potentially limiting its application in field-tests.

Researchers have reported improved sensitivity using Fe₃O₄ magnetic nanoparticles and multifunctional nanospheres in certain applications. However, these approaches typically utilize complicated detecting agent preparations and relatively high associated diagnostic costs. Besides these well adopted clinical diagnostics methods, several bioassays have been reported in current literature, with most of the works focusing on antigen-antibody conjugation or oligonucleotide hybridization and sensing signal transduced through florescence resonance energy transfer, single-particle interferometric reflectance imaging, opto-fluidic nanoplasmonic biosensors, nanoantenna array, memristor and field-effect transistors. Some of these methods demonstrate very sensitive detection (limit of detection reaching femtomolar) attributed to delicate signal transduction mechanism. On the other hand, such high sensitivity is often at the cost of elaborate sample preparation and complicated characterization, creating challenges for low cost miniaturized POC applications.

Accordingly, there is a need for additional methods, and related aspects, of detecting SARS-CoV-2 that are low cost, sensitive, easy-to-use, and which yield rapid POC results, particularly under low resource conditions.

### SUMMARY

The present disclosure relates, in certain aspects, to ultra-sensitive gold nanoparticle (AuNP) and/or other plasmonic metal nanoparticle (MNP)-based colorimetric assays for portable and early-stage severe acute respiratory syndrome coronavirus-2 (SARS-CoV-2) detection. In some embodiments, the AuNP and/or other MNP surfaces are functionalized with high affinity antibodies, nanobodies, and/or other antigen binding portions to SARS-CoV-2 related biomarkers. In these embodiments, the biomarkers generally induce the crosslinking of MNPs leading to large MNP aggregate formation and precipitation, resulting in a decrease of MNP monomer concentration in solution and accordingly, a visible change in solution transparency.

In some embodiments, SARS-CoV-2 receptor-binding domain (RBD) protein is used as a biomarker to be detected. By combining the AuNP and/or other MNP solution-based assay with SARS-CoV-2 early-stage infection biomarker RBD, some embodiments of the present disclosure provide a low cost, ultra-sensitive, colorimetric and easy-to-use SARS-CoV-2 diagnostic assay that can detect RBD concentration down to, for example, about 1.1 μg/ml (10 nM) by naked eye or visual inspection and about 5.8 ng/ml (52.7 pM) by portable UV-visible spectrometer detection, which can be readily applied in SARS-CoV-2 point-of-care diagnostics, especially in underdeveloped regions. These and other aspects will be apparent upon a complete review of the present disclosure, including the accompanying figures.

In one aspect, the present disclosure provides a method of detecting a virus in a sample. The method includes contacting the sample with a plurality of gold nanoparticles (AuNPs) and/or other plasmonic metal nanoparticles (MNPs) that are conjugated with one or more antibodies, or antigen binding portions thereof, that bind to an epitope of a viral antigen of or from the virus under conditions sufficient for the one or more antibodies, or the antigen binding portions thereof, to bind to the epitope of the viral antigen of or from the virus in the sample to produce bound viral antigen. The method also includes detecting the viral antigen of or from the virus when one or more aggregations of the bound viral antigen form with one another, thereby detecting the virus in the sample. Related reaction mixtures, devices, kits, and systems are also provided.

In another aspect, the present disclosure provides a method of detecting severe acute respiratory syndrome coronavirus-2 (SARS-CoV-2) in a sample. In some embodiments, the method includes contacting the sample with a plurality of gold nanoparticles (AuNPs) and/or other plasmonic metal nanoparticles (MNPs) that are conjugated (e.g., via a linker in some embodiments) with at least two sets of antibodies (e.g., monoclonal antibodies or the like), or antigen binding portions thereof, in which at least a first set of antibodies, or antigen binding portions thereof, binds to a first epitope of a SARS-CoV-2 protein (e.g., a receptor-binding domain (RBD)) and in which at least a second set of antibodies, or antigen binding portions thereof, binds to a second epitope of a SARS-CoV-2 protein (e.g., the same SARS-CoV-2 protein comprising the first epitope or a different SARS-CoV-2 protein) under conditions sufficient for the first and second set of antibodies, or the antigen binding portions thereof, to bind to the first and second epitopes of the SARS-CoV-2 proteins in the sample to produce bound SARS-CoV-2 proteins. In some embodiments, the method includes contacting the sample with a plurality of gold nanoparticles (AuNPs) and/or other plasmonic metal nanoparticles (MNPs) that are conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins. In some embodiments, the other MNPs comprise silver, copper, aluminum, platinum, palladium, or the like. In some embodiments, the sets of antibodies, or antigen binding portions thereof, comprise nanobodies. In some embodiments, a given antibody, or antigen binding portions thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa). In some embodiments, a given antibody, or antigen binding portions thereof, comprises an equilibrium dissociation constant (K_{D}) on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM). The method also includes detecting the SARS-CoV-2 proteins when one or more aggregations of the bound SARS-CoV-2 proteins form with one another, thereby detecting the SARS-CoV-2 in the sample.

In some embodiments, the detection step comprises determining a change in absorbance at a resonance wavelength of the AuNPs and/or other MNPs. In some embodiments, the method includes quantifying an amount of the SARS-CoV-2 proteins and/or the SARS-CoV-2 in the sample. In some embodiments, the method further includes centrifuging the aggregations of the bound SARS-CoV-2 proteins prior to and/or during the detecting step. In some embodiments, the method further includes freezing the aggregations of the bound SARS-CoV-2 proteins prior to the detecting step. In some embodiments, the method includes drop casting the aggregations of the bound SARS-CoV-2 proteins prior to the detecting step. In some embodiments, the method includes obtaining the sample from a subject. In some embodiments, the method includes administering one or more therapies to the subject when the SARS-CoV-2 is detected in the sample. In some embodiments, the method includes detecting the SARS-CoV-2 within about 20 minutes or less of obtaining the sample from the subject. In some embodiments, the method includes repeating the method using one or more longitudinal samples obtained from the subject (e.g., to monitor a course of therapy and/or progression of the SARS-CoV-2 disease over time).

Essentially any sample type is used in performing the methods disclosed herein. In some embodiments, for example, the sample comprises blood, plasma, or serum. In some embodiments, the sample comprises saliva or sputum. In some embodiments, the sample comprises urine.

In some embodiments, the detecting step comprises measuring a colorimetric change when the one or more aggregations of the bound SARS-CoV-2 proteins form with one another. In some embodiments, the method includes visually detecting the colorimetric change when the one or more aggregations of the bound SARS-CoV-2 proteins form with one another. In some embodiments, the method includes detecting the colorimetric change when the one or more aggregations of the bound SARS-CoV-2 proteins form with one another using a spectrometer. In some embodiments, a concentration of SARS-CoV-2 proteins in the sample is about 15 nM or less. In some embodiments, a concentration of SARS-CoV-2 proteins in the sample is about 100 pM or less. In some embodiments, the AuNPs and/or other MNPs comprise a substantially spherical shape. In some embodiments, the AuNPs and/or other MNPs comprise a cross-sectional dimension of between about 10 nm and about 100 nm.

In another aspect, the present disclosure provides a reaction mixture that includes a sample comprising severe acute respiratory syndrome coronavirus-2 (SARS-CoV-2) protein, and a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein in the sample; or (ii) conjugated with a single set of antibodies, or antigen binding portions thereof, that bind to an identical epitope from different monomers of a dimerized SARS-CoV-2 protein in the sample.

In another aspect, the present disclosure provides a composition that comprises a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein in the sample; or (ii) conjugated with a single set of antibodies, or antigen binding portions thereof, that bind to an identical epitope from different monomers of a dimerized SARS-CoV-2 protein in the sample.

In another aspect, the present disclosure provides a device that includes at least one reaction chamber (e.g., a body structure comprising an array of wells or the like) or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein when the reaction chamber or substrate receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins and one or more aggregations of the bound SARS-CoV-2 proteins to produce a colorimetric change in the reaction chamber; or (ii) conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins and one or more aggregations of the bound SARS-CoV-2 proteins to produce a colorimetric change in the reaction chamber. In some embodiments, the one or more aggregations of the bound SARS-CoV-2 proteins are drop cast in or on the reaction chamber or substrate. In some embodiments, a kit includes the device.

In another aspect, the present disclosure provides a system that includes a device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein when the reaction chamber receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins; or (ii) conjugated with a single set of antibodies, or antigen binding portions thereof, that bind to an identical epitope from different monomers of a dimerized SARS-CoV-2 protein when the reaction chamber receives a sample that comprises the SARS-CoV-2 under conditions sufficient for the antibodies, or the antigen binding portions thereof, to bind to the identical epitope from the different monomers of the dimerized SARS-CoV-2 protein in the sample to produce bound SARS-CoV-2 proteins. The system also includes an electromagnetic radiation detection apparatus positioned, or positionable, within sufficient proximity to the device to detect one or more colorimetric changes produced in or on the reaction chamber or substrate when one or more aggregations of the bound SARS-CoV-2 proteins form with one another in or on the reaction chamber or substrate. In some embodiments, the electromagnetic radiation detection apparatus comprises a spectrometer. In some embodiments, the electromagnetic radiation detection apparatus comprises a microscope. In some embodiments, the electromagnetic radiation detection apparatus comprises a light-emitting diode (LED) that transmits light into and/or through the reaction chamber or substrate and a photodetector that detects light from the reaction chamber or substrate. In some embodiments, the system includes a device holder that is structured to hold the device, which device holder comprises at least one optical channel through which light is transmitted.

### BRIEF DESCRIPTION OF THE DRAWINGS

The accompanying drawings, which are incorporated in and constitute a part of this specification, illustrate certain embodiments, and together with the written description, serve to explain certain principles of the methods, reaction mixtures, devices, kits, and related systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of detecting SARS-CoV-2 according to some aspects disclosed herein.

FIG. 2 is a flow chart that schematically shows exemplary method steps of detecting SARS-CoV-2 according to some aspects disclosed herein.

FIG. 3 is a schematic diagram of a work flow for taking visual and spectral measurements of SARS-CoV-2 in samples according to one exemplary embodiment.

FIGS. 4A-D show aspects of the identification and characterization of antigen-specific nanobody binders. (a) Binding of single phage clones to antigen measured by ELISA. Biotinylated RBD proteins were immobilized on streptavidin-coated plates, respectively. BSA was used as a control. (b) BLI analysis of specific binders binding to RBD at different concentrations. The RBD proteins were immobilized on streptavidin biosensors (SA). Measured data were globally fitted. (c) Co-binder validation by sandwich ELISA. The first biotinylated nanobody binders (RBD 10) were immobilized on a plate, incubated with or without antigen, and then the second binders (RBD 8) were detected by a horseradish peroxidase-conjugated antibody. (d) Co-binder validation by two-step binding characterization using BLI. Biotinylated RBD proteins were immobilized on SA sensors. Epitope binning was performed by first dipping into the first binder well for 750 s for saturation and then incubation with second binder.

FIGS. 5A-G show rapid and electronic detection of SARS-CoV-2 RBD using co-binder antibody RBD10/RBD8 with improved sensing performance. (a) Extinction spectra of AuNPs in PDMS well plate. (b-c) Electronic detection system: (b) Schematic and (c) Visual image showing a LED circuit, a photodiode circuit, and a 3D printed Eppendorf tube holder. (d) Spectrometric and electronic readout of RBD signals in 5% HPS. Black solid squares: Extinction peak values (559 nm) extracted from (a) and plotted against RBD concentration. Solid triangles: measured electronic voltage signals directly from microcentrifuges tubes.

FIGS. 6A-D show co-binders RBD8/RBD10 for rapid RBD detection in different buffers. Optical sensing (extracted extinction peak values, in black) and electronic sensing (readout voltage, in dashed) to detect RBD from 1 pM to 1 μM. (a) RBD spectrometric detection in 1×PBS buffer. (b). RBD spectrometric detection in 5% FBS buffer. (c) RBD detection in 5% HPS buffer, with spectrometric readout (black squares, fitted to solid line s) and electronic readout (triangles, fitted to dash lines). (d) RBD spectrometric detection in 5% WB buffer.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, reaction mixtures, devices, kits, and systems, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a SARS-CoV-2 protein, such as a receptor-binding domain (RBD) protein, e.g., a molecule in which one or more immunoglobulin chains is not full length, but which specifically binds to a SARS-CoV-2 protein. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Bind: As used herein, “bind,” in the context of pathogen detection, refers to a state in which a first chemical structure (e.g., a pathogenic particle) is sufficiently associated a second chemical structure such that the association between the first and second chemical structures can be detected.

Conjugate: As used herein, “conjugate” refers to a reversible or irreversible connection between two or more substances or components. In some embodiments, for example, gold nanoparticles (AuNPs) and/or other plasmonic metal nanoparticles (MNPs) are connected to antibodies and/or to antigen binding portions thereof. In some embodiments, AuNPs and/or other plasmonic metal nanoparticles (MNPs) are conjugated with antibodies and/or to antigen binding portions thereof via one or more linker compounds.

Detect: As used herein, “detect,” “detecting,” or “detection” refers to an act of determining the existence or presence of one or more target analytes (e.g., a SARS-CoV-2 protein) and/or a pathogen (e.g., a SARS-CoV-2) in a sample.

Epitope: As used herein, “epitope” refers to the part of an antigen (e.g., a SARS-CoV-2 protein) to which an antibody and/or an antigen binding portion binds.

Reaction Mixture: As used herein, “reaction mixture” refers a mixture that comprises molecules and/or reagents that can participate in and/or facilitate a given reaction or assay. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” means anything capable of being analyzed by the methods, devices, and/or systems disclosed herein.

Severe Acute Respiratory Syndrome Coronavirus-2: As used herein, “severe acute respiratory syndrome coronavirus-2” or “SARS-CoV-2” refers to the coronavirus that emerged in 2019 to cause a human pandemic of an acute respiratory disease, now known as coronavirus disease 2019 (COVID-19).

Specifically Bind: As used herein, “specifically bind,” in the context of pathogen detection, refers to a state in which substantially only target chemical structures (e.g., target SARS-CoV-2 proteins) are sufficiently associated with a corresponding or cognate binding agent (e.g., an antibody, or antigen binding portion thereof), to the exclusion of non-target chemical structures, such that the association between the target chemical structures and the binding agent can be detected.

Subject: As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

Treat: As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

### DETAILED DESCRIPTION

The new coronavirus disease (COVID-19), caused by the RNA virus severe acute respiratory syndrome coronavirus 2 (SARS-CoV-2), in the U.S. alone has infected more than 5.6 million people, caused ˜175,000 deaths, and resulted in loss of more than 10 million jobs and trillions of dollars. Serology tests that detect antibodies responsive to the infection have emerged as a valuable tool to assist virus surveillance, assess the risks of infection, evaluate the quality of convalescent plasma donation, and study the duration and magnitude of immune response post-infection. Many of the available tests are lateral flow assays (LFA) or enzyme-linked immunosorbent assays (ELISA). LFA provides simple positive or negative results, and are feasible for point-of-care (POC) use but less useful to identify the amount, type, or function of the antibodies. ELISA provides a better quantification, but it is not suitable for rapid testing due to its complexity in operation and requirement of laboratory instruments.

SARS-CoV-2 virions are spherical nanoparticles of about 100 nm with a membrane envelope that is studded with homotrimers of the spike (S) glycoprotein. S proteins are post-translationally cleaved in the secretory pathway to yield N- and C-terminal S1 and S2 subunits, respectively. S1 is organized into an N-terminal domain (NTD), a central receptor-binding domain (RBD), and a C-terminal domain (CTD). The S1 RBD engages the viral receptor, human angiotensin-converting enzyme 2 (ACE2), at the host cell surface, followed by S protein cleavage by the transmembrane protease serine protease-2 (TMPRSS2) at the cell surface, as well as in endosomes. This cleavage activates S2 conformational rearrangements that catalyze the fusion of viral and cellular membranes and escape of the viral genome into the cytoplasm, which initiates disease-causing cycles of viral replication. Following infection, most individuals will develop an immune response to the virus, including the production of neutralizing antibodies that can prevent future infection by blocking the binding activity of the S glycoprotein. Therefore, the S glycoprotein is the major antigenic target on the virus for protective antibodies and is thus of high significance for diagnostics as well as the development of vaccines and therapeutic antibodies. Current COVID-19 diagnosis is mainly based on epidemiological history, clinical manifestations and biomolecular marker detection. At present, real-time quantitative polymerase chain reaction (RT-qPCR) that identifies the viral RNA SARS-CoV-2 is most widely used. Yet, the PCR assay has significant limitations, including high cost and slow time-to-answer.

Embodiments of the present disclosure provide plasmonic metal nanoparticle (MNP) based colorimetric assays to identify and quantify COVID19-related antigens using optical and electronic readouts. Analytes (e.g., SARS-CoV-2 proteins, such as RBD proteins) modulate the extent of MNP clustering and precipitation, and accordingly, changes the suspension color and intensity, which can be quantified to determine the concentration, binding affinity, and even binding epitope of the analyte. This disclosure has the capability to substantially promote the availability of serology tests and assist the diagnosis, vaccination, and treatment of COVID-19 disease.

Some embodiments of the present disclosure provide a portable colorimetric sensor design for rapid detection of COVID-19 antigens, including SARS-CoV-2 proteins, such as RBD proteins. In some embodiments, different assay variants can be used, including MNP in suspension and dried states (bare-eye readout), spectroscopic quantification, and optical and structural analysis. In some embodiments, the MNP shape and size, analyte and MNP concentration, and binding affinity affect the limit of detection, dynamic range, and assay time will be incorporated into the assays of the present disclosure. Additionally, antibodies, or antigen binding portions thereof, (e.g., monoclonal antibodies, nanobodies, and/or the like) that bind to epitopes of SARS-CoV-2 proteins can be conjugated on MNPs of different geometries and materials that display distinct colors. Such heterogeneous MNPs can be used to establish a sandwich-type assay capable of detecting multiple types of antigens by bare eyes. As would be recognized by one of ordinary skill in the art based on the present disclosure, the compositions, reaction mixtures, kits, devices, assays, and systems described herein can be used with any SARS-CoV-2 antigen in a given sample recognized by antibodies, or antigen binding portions thereof, conjugated with the MNPs.

Embodiments of the present disclosure also include a new plasmonic metal nanoparticle (MNP) based colorimetric assay platform that will support a variety of sensing schemes, including multiplexed detection of SARS-CoV-2 proteins. Using different assay variants, such as MNP in suspension (e.g., in microcentrifuge tubes or customized PDMS well plate) and dried states (e.g., on glass or gold surface), structural analysis and optical detection are combined with intuitive physical pictures and a theoretical mathematical model to comprehensively understand the mechanisms of MNP-based multivalent analyte-binding. Such studies provide a foundation to further incorporate heterogeneous MNPs displaying distinct colors from blue to red to improve specificity, achieve multiplexed detection, and expand assay functionalities. In addition, portable and inexpensive detecting instruments as disclosed herein provide more precise quantification than bare-eye readout, feasible for clinical settings and field deployment in some embodiments.

In some embodiments, the assays disclosed herein can deliver accurate detection results in about 20 minutes or less by accelerating AuNP and/or other MNP crosslinking, for example, using centrifuge concentration. Multiple characterization methods, including scanning electron microscopy (SEM) and dark field scattering imaging, among other techniques, can be applied for quantitative analysis in SARS-CoV-2 protein detection with less than one nM sensitivity and accuracy. In some embodiments, the present disclosure also demonstrates the feasibility of detecting SARS-CoV-2 protein in serum or other sample types with miniaturized portable UV-visible spectrometers for point-of-care detection. In some embodiments, for example, the MNP solution-based colorimetric assays and other aspects disclosed herein provide ultra-high sensitivity, low cost and electricity free colorimetric detection, which can be readily utilized for point-of-care detection of SARS-CoV-2 in remote pandemic regions. These and other aspects will be apparent upon a complete review of the present disclosure, including the accompanying figures.

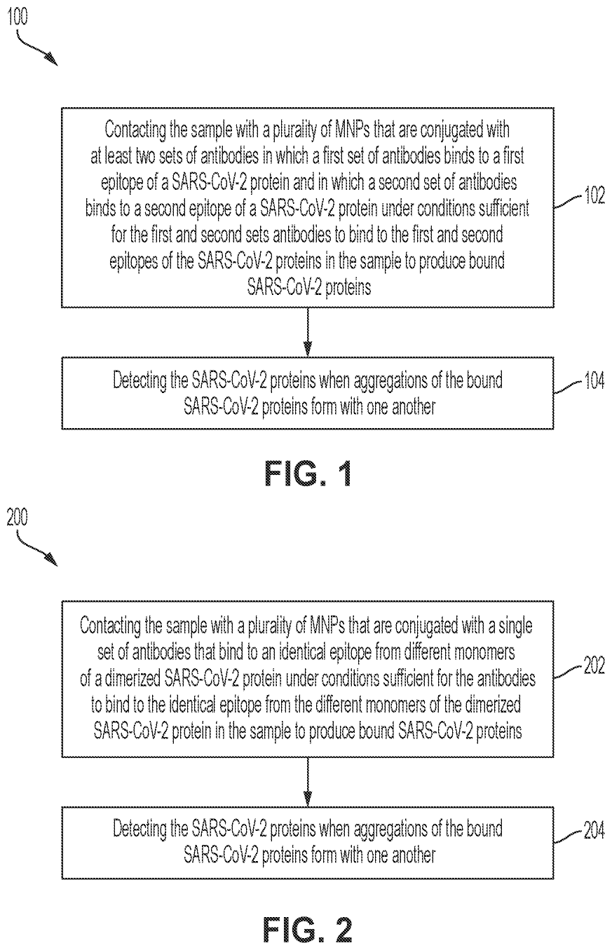

To illustrate, FIG. 1 is a flow chart that schematically shows exemplary method steps of detecting severe acute respiratory syndrome coronavirus-2 (SARS-CoV-2) in a sample according to some embodiments. As shown, method 100 includes contacting the sample with a plurality of plasmonic metal nanoparticles (MNPs) (e.g., gold nanoparticles (AuNPs) or the like) that are conjugated with at least two sets of antibodies (or antigen binding portions thereof, (e.g., monoclonal antibodies, nanobodies, etc.)) in which a first set of antibodies (or antigen binding portions thereof) binds to a first epitope of a SARS-CoV-2 protein and in which a second set of antibodies (or antigen binding portions thereof) binds to a second epitope of a SARS-CoV-2 protein under conditions sufficient for the first and second set of antibodies (or the antigen binding portions thereof) to bind to the first and second epitopes of the SARS-CoV-2 proteins in the sample to produce bound SARS-CoV-2 proteins (step 102).

In another embodiment, FIG. 2 provides a flow chart that schematically shows other exemplary method steps of detecting SARS-CoV-2. As shown, step 202 of method 200 includes contacting the sample with a plurality of MNPs (e.g., gold nanoparticles (AuNPs) or the like) that are conjugated with a single set of antibodies (or antigen binding portions thereof (e.g., monoclonal antibodies, nanobodies, etc.)) that bind to an identical epitope from different monomers of a dimerized SARS-CoV-2 protein under conditions sufficient for the antibodies (or the antigen binding portions thereof) to bind to the identical epitope from the different monomers of the dimerized SARS-CoV-2 protein in the sample to produce bound SARS-CoV-2 proteins. Essentially any sample type is used in performing method 100 or method 200. In some embodiments, for example, the sample comprises blood, plasma, or serum. In some embodiments, the sample comprises saliva or sputum. In some embodiments, the sample comprises urine. In some embodiments, a given antibody, or antigen binding portions thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa). In some embodiments, a given antibody, or antigen binding portions thereof, comprises an equilibrium dissociation constant (K_{D}) on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM). The production of antibodies and antigen binding portions thereof suitable for use with methods, devices, and other aspects of the present disclosure are described further herein or otherwise know to a person having ordinary skill in the art.

Methods 100 and 200 each also include detecting the SARS-CoV-2 proteins when aggregations of the bound SARS-CoV-2 proteins form with one another to thereby detect the SARS-CoV-2 in the sample (step 104 or 204). In some embodiments, the detection step includes determining a change in absorbance at a resonance wavelength of the MNPs. In some embodiments, method 100 or 200 includes quantifying an amount of the SARS-CoV-2 proteins and/or the SARS-CoV-2 in the sample. In some embodiments, method 100 or 200 further includes centrifuging the aggregations of the bound SARS-CoV-2 proteins prior to and/or during the detecting step. In some embodiments, method 100 or 200 further includes freezing the aggregations of the bound SARS-CoV-2 proteins prior to the detecting step. In some embodiments, method 100 or 200 includes drop casting the aggregations of the bound SARS-CoV-2 proteins prior to the detecting step. In some embodiments, method 100 or 200 includes obtaining the sample from a subject. In some embodiments, method 100 or 200 includes administering one or more therapies to the subject when the SARS-CoV-2 is detected in the sample. In some embodiments, method 100 or 200 includes detecting the SARS-CoV-2 within about 20 minutes or less of obtaining the sample from the subject. The method 100 or 200 may detect the SARS-CoV-2 within 25 minutes, 24 minutes, 23 minutes, 22 minutes, 21 minutes, 20 minutes, 18 minutes, 16 minutes, 14 minutes, 12 minutes, 10 minutes, 8 minutes, 6 minutes, 5 minutes, 4 minutes, 3 minutes, 2 minutes 1 minute, or any range between these values. In some embodiments, method 200 includes repeating the method using one or more longitudinal samples obtained from the subject (e.g., to monitor a course of therapy and/or progression of the SARS-CoV-2 disease over time). In some embodiments, the detecting step comprises measuring a colorimetric change when the one or more aggregations of the bound SARS-CoV-2 proteins form with one another. In some embodiments, method 100 or 200 includes visually detecting the colorimetric change when the one or more aggregations of the bound SARS-CoV-2 proteins form with one another. In some embodiments, method 100 or 200 includes detecting the colorimetric change when the one or more aggregations of the bound SARS-CoV-2 proteins form with one another using a spectrometer. In some embodiments, a concentration of SARS-CoV-2 proteins in the sample is about 15 nM or less (e.g., when visually detecting the colorimetric change). In some embodiments, a concentration of SARS-CoV-2 proteins in the sample is about 100 pM or less (e.g., when detecting the colorimetric change using a spectrometer). In some embodiments, the MNPs (e.g., AuNPs and/or the like) comprise a substantially spherical shape. In some embodiments, the MNPs comprise a cross-sectional dimension of between about 10 nm and about 100 nm.

Some aspects of the present disclosure include a colorimetric sensing mechanism that uses nanobody-coated metal nanoparticles for SARS-CoV-2 detection. Some of these embodiments include synthesizing nanobodies (e.g., RBD8 and RBD10, as described further herein) for the COVID receptor-binding domain (RBD) protein. Nanoparticles are optionally prepared by producing the nanobodies in bacterial host cells, biotinylating the nanobodies, and coating AuNPs with the biotinylated nanobodies. Aggregations of bound RBD proteins are then detected when samples are contacted with the nanobody coated AuNPs in some embodiments. Some embodiments include the use of a portable readout system, which includes a quantitative electronic readout with light-emitting diodes (LEDs) and photodetectors, bare-eye colorimetric readout, and a quantitative spectroscopic analysis using a polydimethylsiloxane (PDMS) well plate and a spectrometric detector.

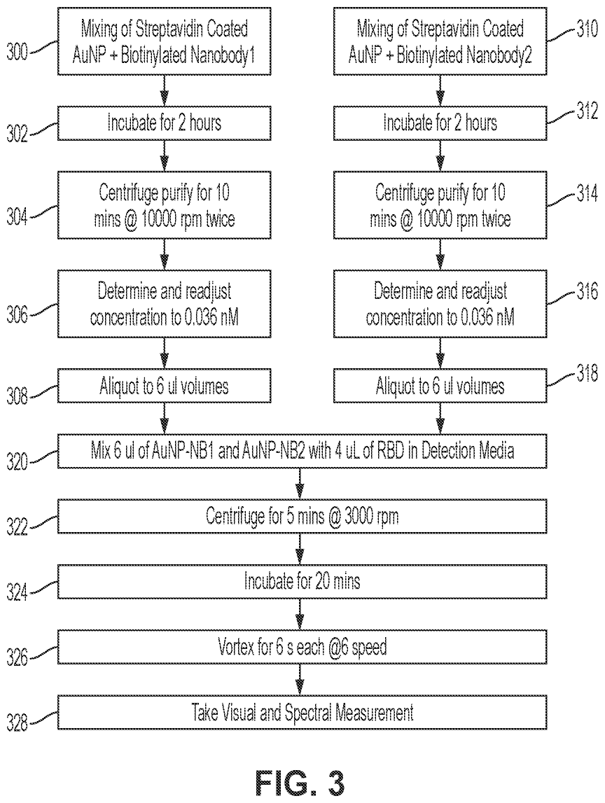

FIG. 3 is a schematic diagram of a work flow for taking visual and spectral measurements of SARS-CoV-2 in samples according to one exemplary embodiment. As shown, the work flow includes mixing streptavidin coated AuNPs with biotinylated nanobody 1 (e.g., a nanobody that binds to a first epitope of RBD) (step 300), incubation for two hours (step 302), centrifugation-based purification for 10 minutes at 10000 rpm performed two times (step 304), determining and readjusting the concentration to 0.036 nM (step 306), and aliquoting 6 μL volumes (step 308). The work flow also includes mixing streptavidin coated AuNPs with biotinylated nanobody 2 (e.g., a nanobody that binds to a second epitope of RBD) (step 310), incubation for two hours (step 312), centrifugation-based purification for 10 minutes at 10000 rpm performed two times (step 314), determining and readjusting the concentration to 0.036 nM (step 316), and aliquoting 6 μL volumes (step 318). The 6 μL volumes from steps 308 and 318 are mixed with a 4 μL sample comprising RBD in a detection media (step 320), which is then centrifuged for 5 minutes at 3000 rpm (step 322), incubated for 20 minutes (step 324), and vortexed for 6 seconds (step 326). Visual and spectral measurements are then taken in step 328.

In some embodiments, the present disclosure provides a composition that comprises a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein in the sample; or (ii) conjugated with a single set of antibodies, or antigen binding portions thereof, that bind to an identical epitope from different monomers of a dimerized SARS-CoV-2 protein in the sample.

In another aspect, the present disclosure provides a reaction mixture that includes a sample comprising SARS-CoV-2, and a plurality of MNPs (e.g., gold nanoparticles (AuNPs)) that are (i) conjugated with at least two sets of antibodies, or antigen binding portions thereof, (e.g., nanobodies, etc.) wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein in the sample; or (ii) conjugated with a single set of antibodies, or antigen binding portions thereof, that bind to an identical epitope from different monomers of a dimerized SARS-CoV-2 protein in the sample.

In another aspect, the present disclosure provides a device that includes at least one reaction chamber (e.g., a body structure comprising an array of wells or the like) or substrate comprising a plurality of MNPs (e.g., gold nanoparticles (AuNPs) and/or other MNPs) that are conjugated with at least two sets of antibodies, or antigen binding portions thereof, wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein when the reaction chamber or substrate receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins and one or more aggregations of the bound SARS-CoV-2 proteins to produce a colorimetric change in the reaction chamber; or (ii) conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins and one or more aggregations of the bound SARS-CoV-2 proteins to produce a colorimetric change in the reaction chamber. In some embodiments, the one or more aggregations of the bound SARS-CoV-2 proteins are drop cast in or on the reaction chamber or substrate. In other embodiments, the one or more aggregations of the bound SARS-CoV-2 proteins may be deposited in or on the reaction chamber or substrate in a variety of deposition techniques. The deposition technique may be spin coating, dip coating, spray coating or any other similar technique known to one of skill in the art. In some embodiments, a kit includes the device.

In another aspect, the present disclosure provides a system that includes a device, comprising at least one reaction chamber or substrate comprising a plurality of gold nanoparticles (AuNPs) and/or other MNPs (e.g., MNPs that comprise silver, copper, aluminum, platinum, palladium, or the like) that are (i) conjugated with at least two sets of antibodies, or antigen binding portions thereof, wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein when the reaction chamber receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins; or (ii) conjugated with a single set of antibodies, or antigen binding portions thereof, that bind to an identical epitope from different monomers of a dimerized SARS-CoV-2 protein when the reaction chamber receives a sample that comprises the SARS-CoV-2 under conditions sufficient for the antibodies, or the antigen binding portions thereof, to bind to the identical epitope from the different monomers of the dimerized SARS-CoV-2 protein in the sample to produce bound SARS-CoV-2 proteins. The system also includes an electromagnetic radiation detection apparatus positioned, or positionable, within sufficient proximity to the device to detect one or more colorimetric changes produced in or on the reaction chamber or substrate when one or more aggregations of the bound SARS-CoV-2 proteins form with one another in or on the reaction chamber or substrate. In some embodiments, the electromagnetic radiation detection apparatus comprises a spectrometer. In some embodiments, the electromagnetic radiation detection apparatus comprises a microscope. Exemplary devices and systems are described further herein.

### Example

### Synthetic Nanobody-Functionalized Nanoparticles for Accelerated Development of Rapid, Accessible Detection of Viral Antigens

### Introduction

Successful control of emerging infectious diseases requires accelerated development of fast, affordable, and accessible assays to be widely implemented at a high frequency. Here we present a generalizable assay platform, nanobody-functionalized nanoparticles for rapid, electronic detection (Nano2RED), demonstrated in the detection of Ebola and COVID-19 antigens. To efficiently generate high-quality affinity reagents, synthetic nanobody co-binders and mono-binders with high affinity, specificity, and stability were selected by phage display screening of a vastly diverse, rationally randomized combinatorial library, bacterially expressed and site-specifically conjugated to gold nanoparticles (AuNPs) as multivalent in-solution sensors. Without requiring fluorescent labelling, washing, or enzymatic amplification, these AuNPs reliably transduce antigen binding signals upon mixing into physical AuNP aggregation and sedimentation processes, displaying antigen-dependent optical extinction readily detectable by spectrometry or simple electronic circuitry. With nanobodies against an Ebola virus secreted glycoprotein (sGP) and a SARS-CoV-2 spike protein receptor binding domain (RBD) as targets, Nano2RED showed a high sensitivity (limit of detection of ˜10 μg/mL for sGP and ˜40 μg/mL for RBD in diluted human serum), a high specificity, and a large dynamic range (˜7 logs). Unlike conventional assays where slow mass transport for surface binding limits the assay time, Nano2RED features fast antigen diffusion at micrometer scale, and can be accelerated to deliver results within a few minutes. The rapid detection, low material cost (estimated <$0.01 per test), inexpensive and portable readout system (<$5 and <100 cm³), and digital data output, make Nano2RED particularly suitable for screening of patient samples with simplified operation and accelerated data transmission. Our method is widely applicable for prototyping diagnostic assays for other antigens from new emerging viruses.

In recent times, we have witnessed the emergence of many infectious viral diseases, from the highly fatal Ebola virus disease (EVD, with a fatality rate of 45% to 90%) to the highly contagious coronavirus disease 2019 (COVID-19) with its global >200 million infections and >4 million deaths as of August 2021. Future emergence of Disease X, as contagious as COVID-19 and as lethal as EVD, would pose an even greater threat to humanity, and will be both difficult to prevent or predict. During disease emergence, early pathogen identification and infection isolation are important for containing disease transmission. Therefore, for effective mitigation, it is necessary to accelerate the design, development, and validation of diagnostic processes, as well as to make the diagnostic tools broadly accessible within weeks of the initial outbreak.

Current diagnostic methods rely on the detection of the genetic (or molecular), antigenic, or serological (antibody) markers. Genetic diagnostics use DNA sequencing, polymerase amplification assays, or most recently, CRISPR technologies. For example, real-time reverse transcription polymerase chain reaction (RT-PCR) tests are viewed as the gold standard for their high sensitivity; however, these tests are also costly, time-consuming, and instrument-heavy. Genetic tests also can often display false positives by picking up genetic fragments from inactive viruses. In comparison, antigen and antibody detections are complementary as they allow more rapid, affordable, and accessible detection without complex sample preparation or amplification. As such, these detection methods are viewed as suitable for surveillance and timely isolation of highly infectious individuals, particularly outside clinical settings. While antibody (e.g., IgM) detection has been used for disease diagnostics, it is less predictive and more suitable for immune response studies. In comparison, viral protein antigen tests provide a reliable field-test solution in diagnosing symptomatic patients, and may serve to screen asymptomatic contacts that may become symptomatic. In addition, since they are rapid, easy to operate, and low-cost, antigen tests can be deployed at high frequencies and large volumes for in-time surveillance, which is thought to be the most important factor in disrupting a virus transmission chain.

Current antigen diagnostics typically employ enzyme-linked immunosorbent assays (ELISA) and lateral flow immunoassays (LFIs). ELISA is the workhorse for analyzing antigens and antibodies, but it requires a multistep workflow and a series of washing steps, hours of incubation prior to readout, and a readout system dependent on substrate conversion and luminescence recording. Deployment of ELISAs in high-throughput mass screenings requires automated liquid handling systems to coordinate the complex workflow, which is not ideal for portable uses. LFIs are potentially much easier to use outside lab settings but usually have much lower sensitivity and thus poorer accuracy compared to ELISA.

Here we report a modular strategy, i.e., nanobody-conjugated nanoparticles for rapid electronic detection (Nano2RED), which can quickly establish a rapid, accessible antigen diagnostic tool within a few weeks of pathogen identification. To generate high-quality affinity reagents within two weeks for any given purified marker protein, we have streamlined a protocol for phage selection of single-domain antibodies (or nanobodies) from a highly diverse combinatorial library (>109) and bacterial expression of top hits with a significantly faster turnaround time and a lower cost than mammalian expression. Unlike traditional antibodies requiring non-specific conjugation to any solid support, potentially resulting in loss of function, nanobodies were genetically fused with an AviTag for site-specific biotinylation and immobilization onto streptavidin-coated gold nanoparticles (AuNPs). These nanobody-functionalized AuNPs serve as multivalent antigen binding sensors in our Nano2RED assays for Ebola and COVID-19 antigen detection. Based on only physical processes, including plasmonic NP color display, NP precipitation, and semiconductor photon absorption, Nano2RED quantitatively transduces antigen binding into colorimetric, spectrometric, and electronic readouts. Without fluorescent labeling or chemiluminescent readout, Nano2RED differs fundamentally from conventional high-sensitivity tests (e.g., genetic tests or ELISA) that are generally expensive and more suitable for lab use. Yet, Nano2RED greatly outperforms conventional portable and low-cost tests (e.g., LFIs), which are not qualitative or sensitive enough. Uniquely, Nano2RED features portability, low cost, and simplicity while preserving a high sensitivity (LOD of ˜ 0.13 pM or 11 μg/mL in Ebola sGP sensing), a high specificity (distinguishing sGP from its membrane-anchored isoform GP1,2) and a large dynamic range (˜7 logs). Additionally, its electronic readout capability can be extended to automate data collection, storage, and analysis, further reducing the workload health care workers, and speeding up diagnostic and surveillance response.

### Nanobody Co-Binder Selection for AuNP Functionalization

We generated nanobody co-binders (i.e., two mono-binders simultaneously binding to non-overlapping epitopes in the same antigen) against target antigens for a new in-solution assay to improve the sensitivity and specificity. Traditional methods for selecting antibody-based co-binders are slow and costly, so here we established a fast, robust protocol including the phage display selection of the combinatorial nanobody library, parallel bacterial protein production, co-binder validation, and AuNP functionalization that can be completed in less than two weeks upon the availability of an antigen protein. Nanobodies, a single-domain (12-15 kDa) functional antibody fragments from camelid comprising a universal scaffold and three variable complementarity-determining regions, are ideally suited for phage display selection and low-cost bacterial production. To avoid relatively lengthy and costly procedures and animal protection issues associated with traditional antibody screening, we screened the synthetic nanobodies library with an optimized thermostable scaffold prepared in our previous work. The Ebola antigen, sGP, is a homodimeric isoform of the glycoproteins encoded by a GP gene of all five species of Ebolavirus with multiple post-translational modifications. sGP is believed to act as a decoy to disrupt the host immune system by absorbing anti-GP antibodies. Given its abundance in the blood stream upon infection and its quantitative correlation with disease progression and humoral response, sGP is widely used as a circulating biomarker in EBOV diagnostics. The chosen SARS-CoV-2 antigen, RBD, engages the viral receptor, human angiotensin-converting enzyme 2 (ACE2), causing conformational changes that trigger a cleavage needed for viral infection. It is a major antigenic target for protective antibodies, and thus is highly significant for diagnostics, as well as for the development of vaccines and therapeutic neutralizing antibodies.

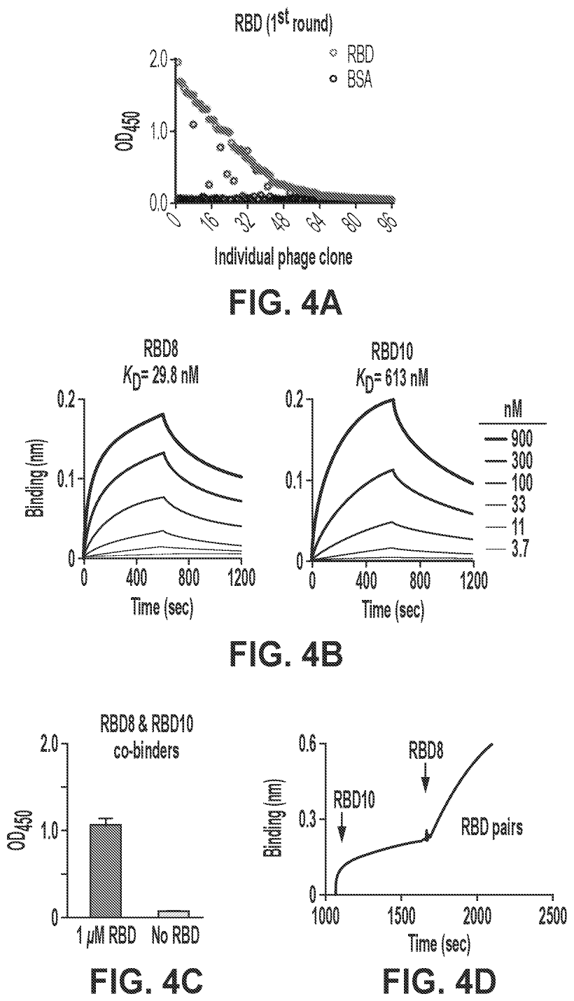

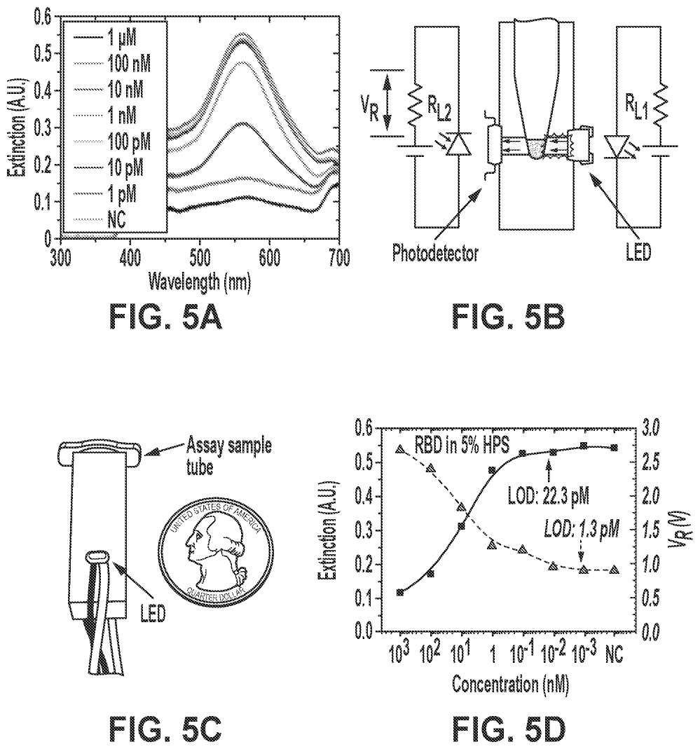

To efficiently identify nanobodies that can bind non-overlapping epitopes of an antigen protein, we assessed clonal diversity and co-binding abilities of candidates enriched in different biopanning rounds. The antigens, sGP (ManoRiver) and RBD (residues 328-531), were expressed as AviTag fusions in HEK293 and biotinylated for immobilization on streptavidin-coated magnetic beads. We identified three co-binder pairs from 10 unique nanobodies out of 96 randomly picked clones that specifically bind to sGP after three rounds of biopanning. For the RBD with a relatively smaller size (˜30 kDa), we identified two co-binder pairs from 12 unique binders after the first round (FIG. 4A). The two top co-binder pairs, termed sGP7-sGP49 and RBD8-RBD10, were bacterially expressed and purified with high yields (1.5 to 6 mg per liter of culture). Their equilibrium dissociation constants (K_{D}) were measured to be in the nanomolar range by Bio-Layer Interferometry (BLI) (FIG. 4B) and the co-binding activities were validated by ELISA (FIG. 4C) and BLI (FIG. 4D). Lastly, nanobodies were biotinylated with E. coli biotin ligase (BirA) as previously reported and then loaded to streptavidin-coated AuNPs (see Methods section).

### Nanobody-Functionalized Nanoparticles for Sensing

In our assay design, AuNPs densely coated with biotinylated nanobodies allow multivalent antigen sensing known to significantly enhance antigen binding compared to the monovalent binding. Further, the multivalent binding also facilitates AuNP aggregation at the presence of the antigen and subsequent precipitation, producing antigen-concentration-dependent signals within minutes. The AuNP aggregation is further quantified by optical and electronic measures. In our sensing scheme, AuNPs, without nonspecific particle-particle interaction, are initially homogenously dispersed in colloid, presenting a reddish color from characteristic localized surface plasmon resonance (LSPR) extinction. Upon mixing with viral antigens, multiple AuNPs are pulled together by the antigen-nanobody binding to gradually form large aggregates. Compared to a single AuNP, the formation of AuNP aggregates gradually shifts LSPR extinction to higher wavelengths with broadened resonance attributed to plasmonic coupling between AuNPs, a phenomenon that can be simulated by finite-difference time-domain (FDTD) method. This leads to increased transparency of the AuNP colloid preciously described in DNA and protein sensing applications. Large AuNP aggregates can form pellets as gravity overtakes the fluidic drag force. As a result, decreased AuNP concentrations in the upper liquid result in a colorimetric change correlated with sGP concentrations. The color change can be directly visualized by eye, and quantified in a well plate by spectrophotometer or using a simple electronic device that measures the AuNP extinction.

### Finite-Difference Time-Domain (FDTD) Simulation of AuNP Extinction

We performed FDTD simulation of different numbers of AuNPs in a cluster. AuNP cluster with given number gold nanoparticles were modeled using densely packed 80 nm AuNP nanoparticles with a spacing of 12.8 nm (sGP49-sGP-sGP49 bridge length). The boundary condition along x-, y- and z-direction was set as perfect matched layer (PML). A total-field scattered-field (TFSF) light source (400-1000 nm) was used to calculate extinction cross section. The mesh size was set to be 5 nm in x-, y- and z-direction in the AuNP cluster region. Six monitors recording the power flux were set outside TFSF source region normal to x-, y- and z-directions. The background index was set as 1.33 to simulate the solution environment. The simulation time was set to 5000 fs and auto shut off threshold was set as 5×10⁶.

We found the resonance peaks red-shift for small clusters, but the resonance becomes less evident for even larger clusters, e.g., more than 10 AuNPs. This effect is expected to be related to inter-particle coupling. It also indicates that the experimentally observed extinction is likely mainly attributed to small clusters and AuNP monomers.

### Colorimetric and Spectrometric Sensing of sGP

The size and shape of AuNPs determine the optical extinction and therefore the suspension color, hence affecting the sensitivity and assay incubation time. Here, the AuNP size effect was studied with NP diameters of 40, 60, 80, and 100 nm in sensing of Ebola sGP proteins using sGP49 nanobody in 1× phosphate buffered saline (PBS) buffer (described further herein). To standardize the measurement, the sGP signals were collected using a UV-visible spectrometer coupled to an upright microscope. We custom-designed a polydimethylsiloxane (PDMS) well plate bonded to glass slides as the sample cuvette. Top-level liquid from sGP sensing samples (5 μL) after incubation were loaded and inspected by optical imaging and spectroscopy readout. Clearly, as evidenced in the optical images, the color of the assay is redder for small NPs but greener for larger ones, which is attributed to a redshift in extinction resonance wavelengths at larger NP sizes. Additionally, a significant color contrast was observed in distinguishing 10 nM and higher sGP concentration from the reference negative control (NC) sample (with only PBS buffer but no sGP) for all AuNP sizes, indicating that sGP can be readily detected by the naked eye. Such colorimetric diagnostics would be very useful for qualitative or semi-quantitative diagnostics in resource-limited settings, but less ideal for quantitative and ultrasensitive detection.

Additional accurate sGP detection was performed by quantifying the AuNP extinction signals in the PDMS plate using our spectroscopic system. The AuNP extinction is correlated with its concentration [NP] and diameter d following σₑₓₜ∝[NP]d³. A decrease of extinction indicated a drop of [NP] in the upper liquid level caused by antigen-induced AuNP precipitation. Further, the AuNP extinction peak values were extracted and plotted as standard curves against the sGP concentration at each AuNP size. This incubation-based assay had a large dynamic range of ˜100 pM to ˜ 100 nM for all AuNP sizes. In addition, the incubation was found to take 4 to 7 hours, using 40 to 100 nm NPs for detecting 10 nM sGP in 1×PBS. This NP size-dependent response could be understood intuitively from the antigen binding dynamics and the AuNP precipitation process. On the one hand, smaller NPs had higher starting concentrations, given that in our design the starting suspension extinction σₑₓₜ∝[NP]d³ was about the same for all sizes, and therefore were expected to initiate the antigen-binding and NP aggregation reaction relatively faster. On the other hand, the precipitation of smaller aggregates took a longer time, resulting in a longer incubation period. From these experimental analyses in 1×PBS buffer, we chose 80 nm AuNPs to further characterize the assay performance in sGP sensing. This selection was based on several factors: their slightly higher sensitivity (˜15 pM, compared to ˜100 pM for other sizes), larger detection dynamic range (up to 4 logs, compared to 2 to 3 logs for other sizes), and shorter incubation time (3 to 4 hours, compared to 4 to 7 hours for other sizes).

To understand the assay's working mechanism, we complemented the solution-phase optical testing by inspecting the AuNP precipitates in solid state using different structural and optical characterization methods (described further herein). First, cryogenic transmission electron microscope (CryoTEM) images showed aggregates of AuNPs formed with 1 nM sGP with an average cluster size of 1.8 by 1.4 μm, while only 80 nm AuNP without clusters were observed in the upper-level liquid or in the precipitates of the NC sample. This supports our sensing mechanism in that AuNP precipitation serves to transduce antigen binding to solution color change for sensing readout. Further, we have performed drop-casting to deposit AuNP upper-level liquid samples on glass slides for optical extinction analysis and on gold films for scanning electron microscopy (SEM) and dark field scattering imaging. The measurement results were, in general, consistent with spectrometric in-solution sGP detection using PDMS well plate, but inferior in sensitivity (150 pM for SEM, ˜174 pM for drop-cast on glass slide, and ˜1 nM for dark filed imaging, Table 1). The decreased sensitivity could be attributed to inherent variations associated with sample preparation and background noise in the readout systems. These solid-state characterization methods were non-ideal for accessible and precise detection, given the low sensitivity and need of lab instrument for readout, but they provided valuable insight into the nano-scale NP aggregation process.

TABLE 1
Performance of the Nano2RED in Ebola sGP protein sensing

| | | | Readout | Readout | | | | | Diagnostic | |
|---|---|---|---|---|---|---|---|---|---|---|
| Assay | Readout | Readout | system | system | | Assay | Nano- | | sensitivity | |
| format | method | system | cost | size | | time | body | Buffer | pM | pg/mL |
| Incubation | Spectrometric | Lab | ~$25,000 | ~3 | m³ | 3-4 h | sGP49 | FBS | 15 | 1250 |
| | Spectrometric | microscope | | | | 3-4 h | | PBS | 174 | 14529 |
| | (cast on glass) | | | | | | | | | |
| | Dark Field | | | | | 3-4 h | | | 1000 | 83500 |
| | (cast on gold) | | | | | | | | | |
| | SEM (cast on | SEM | ~$65,000 | 3-5 | m³ | 3-4 h | | | 150 | 12530 |

| | | | | | | | | | |
|---|---|---|---|---|---|---|---|---|---|
| | gold) | | | | | | | | |
| Centrifuge- | Colorimetric | Eyes or | ~$200 | ~14 × 7 × | 5-20 | sGP49 | FBS | 1000 | 83500 |
| accelerated | | smart phones | (phone) | 0.8 = 78 | min | | | | |
| (rapid) | | | | cm³ | | sGP49/ | FBS | 100 | 8350 |
| | | | | | | sGP7 | | | |
| | Spectrometric | Lab | ~$25,000 | ~3 m³ = | | sGP49 | FBS | 80 | 6680 |
| | | microscope | | 3,000,000 | | sGP49/ | PBS | 0.16 | 13 |
| | | | | cm³ | | sGP7 | FBS | 1.05 | 88 |
| | | | | | | | HPS | 1.26 | 105 |
| | | | | | | | WB | 18.2 | 1520 |
| | | Portable | $1,000 | 8.8 × 6.3 × | | sGP49 | FBS | 99 | 8270 |
| | | spectrometer | | 3.1 = 170 | | | | | |
| | | | | cm³ | | | | | |
| | Electronic | LED & | ~$5 | 1.1 × 1.1 × | | sGP49 | FBS | 27 | 2250 |
| | | photodetector | | 3.3 = 4 | | | | | |
| | | | | cm³ | | sGP49/ | HPS | 0.13 | 11 |
| | | | | | | SGP7 | | | |

sGP was further detected in diluted fetal bovine serum (FBS, 5%) using 80 nm sGP49-functionalized AuNPs. Similarly, to test in 1×PBS, after 3-hour incubation in microcentrifuge tubes, the upper-level liquid samples were loaded into a PDMS well plate and measured by spectrometer. From the plot of extinction peak values against sGP concentration, our assay could again detect sGP over a broad range from 10 pM to 100 nM, which supports clinically relevant Ebola detection from patients' blood (sub-nM to UM). Here the three-sigma limit of detection (LoD), defined as the concentration displaying an extinction differentiable from the NC sample (E_{NC}), or E_{NC}−3σ where σ is the measurement variation of all samples, was found to be about 15 pM (or 1.25 ng/ml), comparable to that measured using sGP49 phage ELISA (LOD estimated ˜80 pM, Table 2). The LOD can be understood from simple and rough estimations based on the nature of multivalent antigen binding (described further herein). We also found the 10 nM sGP could be easily distinguished from NC sample at a broad temperature range from 20 to 70° C. This indicates our assay can be transported, stored, and tested at ambient temperatures without serious concerns of performance degradation, which is very important for mass screening.

TABLE 2
LOD analysis of Ebola sGP and COVID RBD detection with Phage-ELISA

| | | | | σ (A.U.) | | | LOD (pM) | | |
|---|---|---|---|---|---|---|---|---|---|
| Antigen | Binder | Media | Readout method | σ_{PS4} | σ_{PSA} | σ_{NC} | σ_{PS4} | σ_{PSA} | σ_{NC} |
| Ebola | sGP49 | PBS | Colorimetric | 0.00121 | 0.00851 | 0.000622 | 76 | 6200 | 38 |
| sGP | | | | | | | | | |
| COVID- | R8-R10 | PBS | Colorimetric | 0.00415 | 0.0331 | 0.002538 | 30 | 4570 | 1 |
| 19 RBD | | | | | | | | | |

### Impact of Nanoparticle Size: sGP Sensing by Incubation

Here, the AuNP size effect was studied with NP diameters of 40, 60, 80, and 100 nm in sensing of Ebola sGP proteins from 1 pM to 1 μM in 1×PBS buffer. The sGP signals were collected using a UV-visible spectrometer coupled to an upright microscope. We custom designed a polydimethylsiloxane (PDMS) well plate, consisting of 2 mm diameter and 3 mm thick punched holes, that is bonded to a 0.5 mm thick diced fused silica.

Additionally, the AuNP concentrations were adjusted to have roughly identical optical density levels at their peak plasmonic resonance wavelengths (533, 544, 559, and 578 nm for 40, 60, 80, and 100 nm diameter), at an AuNP concentration [NP] of 0.275, 0.086, 0.036, and 0.019 nM, respectively. The extinction coefficient of NPs is theoretically proportional to their total mass (or volume) as σₑₓₜ∝[NP]d³, therefore, [NP] drops with the particle diameter given we intentionally standardize the total extinction of all the NPs.

Here the UV-visible extinction can be mathematically defined as E=log₁₀ (I₀/I)=εcl, where E is the measured AuNP extinction, I₀ and I are the light intensity collected at the reference and sGP sample cuvettes, ε is the AuNP extinction coefficient, c is AuNP concentration, and I is optical path (the solution depth, ˜3 mm here).

We further extracted the extinction peak intensity for each assay sample and plotted the standard curves of extinction versus concentration for each AuNP size. The sGP signal at low concentration (<10 pM) was indistinguishable from the NC signal (with an extinction E_{NC} within the range of 0.4-0.45).

In addition to the detection limits, the assay detection time was studied at 10 nM sGP concentration in 1×PBS. The extinction generally started to drop after 0.5-1.5 hour for all sizes, indicating a stage to initiate aggregate formation and precipitation. Extended incubation led to a nearly linear extinction drop, at a rate of 0.049, 0.071, 0.080, and 0.091 hr⁻¹ for 40, 60, 80, and 100 nm NPs, eventually reaching a stable value after 7, 5.5, 4, and 4.5 hours, respectively.

### Rapid Antigen Detection

The PDMS well plate-based spectrometric measurement required about 3 hours incubation for effective AuNP bridging and precipitation, which is shorter than ELISA and much better than many RT-PCR assays. However, rapid diagnostics, that is, less than 30 minutes, is more desirable for accessible infectious disease diagnosis and control of disease spread. Here, we further studied the sensing mechanism, aiming to reduce the detection time (described further herein). In conventional ELISA assays, the antigen diffusion process is usually the rate-limiting step, since the fluidic transport to a solid surface is ineffective given long diffusion length (millimeter scale liquid depth in well plate) and slow fluidic flow speed at plane surfaces (near zero surface velocity). This leads to slow mass transport and ineffective surface binding, and an accordingly long assay time. Differently, diffusion is no longer the limiting factor in our assay, given the use of NPs in lieu of plane surfaces as reaction sites. The diffusion length is estimated to be about 1 μm due to a high NP concentration (e.g. ˜ 0.036 nM for 80 nm AuNPs). The small size and mass of AuNPs (5×10⁻¹⁵ g) result in a high diffusivity (D_{NP}˜4.2 μm²/s from Stokes-Einstein equation) and a high thermal velocity (estimated 0.028 m/s). All of these features promote effective fluidic transport and antigen binding, with an estimated diffusion time of <1 sec.

We further speculated that the AuNP aggregation and precipitation process could play important roles in determining assay time. Here, we developed a simplified model based on Smoluchowski's coagulation equation to understand the aggregation process. Briefly, an empirical parameter P, which defines the probability of antigen-nanobody binding per collision, has a large impact on the modelled assay time. By comparing to experimentally measured assay incubation time, we found using P=1, that is very high-affinity binding, provides a much better prediction compared to using a smaller P value calculated by the ELISA-measured kinetic constants. This observation is attributed to two factors: the ELISA-measured binding kinetics is strongly affected by the surface-limited diffusion process and could not precisely estimate the true nanobody-antigen binding in solution; and the multivalence of the nanobody-bound AuNPs greatly improves the observed “functional affinity” compared to intrinsic mono-binding affinity. Using this model, it was estimated that the aggregation time constant Tagg as 0.87 hour at 0.036 nM AuNP in detection of 10 nM sGP. Yet Tagg could be greatly reduced by increasing AuNP concentration, for example to 0.024 hour, or 36 times shorter, when using 50 times more concentrated NPs.

On the other hand, as gravitational force overcomes fluidic drag, large clusters precipitate to form sedimentation and continuously deplete AuNPs and sGP proteins in the colloid until reaching equilibrium. The sedimentation time can be estimated using the Mason-Weaver equation by τ_{sed}=z/(s·g) where z is the precipitation path (for example the height of colloid liquid), g is the gravitation constant, and s is the sedimentation coefficient dependent on the physical properties of AuNPs and buffers. Given that z˜3.5 mm for 16 μL liquid in a microcentrifuge tube, we calculated that τ_{sed} decreases from 26 hours for 80 nm AuNPs to 1.0 and 0.3 hours for a 400 nm and 800 nm diameter cluster (comparable to experimentally observed clusters of micrometer size at 1 nM sGP), respectively. The estimated aggregation and precipitation times are consistent with the experimentally observed incubation time (˜3 hours).

For rapid detection, we introduced a centrifugation step (1,200×g, 1 min) after antigen mixing to both enhance the reagents' concentration and decrease the precipitation path. This step concentrated AuNPs at the bottom of microcentrifugation tubes without causing non-irreversible AuNP aggregation, with an estimated z of ˜150 μm as seen from optical image. This corresponds to a roughly >20 times reduction in precipitation path and accordingly τ_{sed}. Additionally, the concentrated AuNPs are confined to an estimated <0.34 μL volume, or ˜50 times concentration increase from original 16 μL colloid liquid, leading to a greatly reduced τ_{agg}, estimated from 0.87 hour to 0.024 hour. These calculations indicate that both the aggregation formation and precipitation of the aggregates can take place in just a few minutes, important to shortening assay time. To experimentally validate the rapid detection concept, the assay colloid was incubated for 20 min after centrifugation and then thoroughly vortexed, which served to re-suspend free AuNPs that could have been physically adsorbed to the tube bottom. Indeed, the increased upper-level assay liquid transparency at higher sGP concentration was distinguished visually for sGP>1 nM. The extinction values of the upper-level liquid were extracted at its peak wavelength (˜559 nm), and plotted against sGP concentration, along with the 3-hour incubation results. The rapid test presented comparable performance in dynamic range and LOD (˜80 pM) compared to incubation. Using 10 nM sGP as the antigen, we found the color contrast was high enough to be immediately resolved by the naked eye after vortex mixing, requiring minimal incubation. Including all of the operation steps for sample collection, pipetting, centrifugation, vortex mixing, and readout, this rapid test scheme can be completed in a few minutes.

### Characterization of sGP Sensing Monobinders

### Preparation of Mono-Binder Surface Functioned AuNP Colloid.

The gold nanoparticles (0.13 nM, 80 μL) that were already surface-functioned with streptavidin were first mixed with an excessive amount of biotinylated sGP49 nanobody (1.2 M, 25 μL). The mixture was then incubated for 2 hours to ensure complete streptavidin-biotin conjugation. Next, the mixture was purified by centrifuge (accuSpin Micro 17, Thermo Fisher) at 10,000 rpm for 10 mins and repeated twice to remove unbounded biotinylated sGP49 nanobody. The purified AuNP colloid was measured by Nanodrop 2000 (Thermo Fisher) to determine the final concentration. The concentration of AuNP in colloid was subsequently adjusted to 0.048 nM and was aliquoted into 12 μL in a 500 μL Eppendorf tube. sGP stock solution (6 μM, in 1×PBS) underwent a 10-fold serial dilution and a 4 μL sGP solution of each concentration (4 pM to 4 μM) was mixed with 12 μL AuNP assay colloid and briefly vortexed (mini vortexer, Thermo Fisher) at 800 rpm for 5 seconds. The buffer used in assay preparation and sGP dilution was prepared by diluting 10×PBS buffer and mixing with glycerol and BSA to reach a final concentration of 1×PBS, 20% v/v glycerol and 1 wt % BSA.

### TEM Inspection.

The assay colloids at the bottom of Eppendorf tube were collected and imaged by cryogenic transmission electron microscope (CryoTEM). Cryogenic sample preparation is known to prevent water crystallization and hence preserve protein structures and protein-protein interaction. Clearly, aggregates of AuNPs formed with 1 μM sGP present in the precipitates with average size of 2.3 by 1.4 μm. The cluster size averaged 1.8 by 1.4 μm with 1 nM sGP. For the NC sample, only 80 nm AuNP monomers but no clusters were observed in the precipitates. The aggregate sizes had a large distribution, probably due to a random aggregation process and sample preparation, and thus was not ideally correlated with the sGP concentration.

### Drop-Casting on Glass Slides for Optical Inspection.

The AuNP assay upper-level liquid (1 μL, sGP mono-binder, 80 nm AuNP, N_{AuNP}=0.036 nM) with sGP from 1 pM to 1 μM in PBS were drop-casted on a 1 mm thick glass slide for colorimetric and spectrometric inspections. It can be observed that the dried sample spots displayed light red color, and their transparency increased from around 10 nM (spot 3) and became readily differentiable to the naked eye at 1 μM (spot 1) compared to the reference NC sample (spot 8). We then measured the extinction spectra of each drop-cast spots and extracted the extinction peak intensity at LSPR resonance. The extinction spectra featured AuNP LSPR peaks, similar to those upper-level liquid measurements in the PDMS well plate, but the peak intensity was about one order of magnitude smaller attributed to a significantly shorter optical path (estimated ˜300 μm) compared to the PDMS well plate (˜3 mm).

These samples were stored at room temperature (25° C.) over 12 weeks and re-inspected, and the optical signals were found in general to be consistent over such an extended period with only slight change. The slight increase in extinction, observed especially at the lower sGP concentration, was possibly due to shrinking of the drop cast spot from dehydration as the sample was exposed to dry air. Nevertheless, this also showed the feasibility of quantitative detection of sGP down to 350 pM LoD with a broad dynamic range in detection (100 pM to 1 μM) using a simple and small solid-state sample carrier.

In comparison, we also performed drop-casting of 60 nm AuNPs on glass slides, which also shows performance similar to the detection in the PDMS well plate.

### Drop-Casting on Gold Film for SEM and Dark-Field Inspection.

Scanning electron microscopy (SEM) was employed to investigate the AuNP aggregation in the upper-level liquid. Here 1 μL of 80 nm AuNP assay colloids (n_{AuNP}=0.036 nM, 1×PBS) with targeted sGP (1 pM to 1 μM) were drop-cast onto an oxygen plasma treated gold surface and subsequently dried in air. Gold surface was selected due to its high electron conductivity that dramatically improves the contrast and resolution in imaging. Only AuNP monomers but no large aggregates were observed from the SEM images, confirming that the majority, if not all, of the aggregates should precipitate at the bottom of the tubes. Further, the 80 nm AuNPs were recognized and counted through image analysis, and their density was statistically determined from ten SEM images (total area 10×8.446×5.913 μm²) at each sGP concentration. Clearly, the AuNP density decreased at a higher sGP concentration, i.e. from 1.97 μm⁻² at about 10 pM to 0.26 μm⁻² at 100 nM and finally saturated to 0.24 μm⁻² at about 1 μM or above, which was in accordance with extinction spectrometric measurements of both upper-level liquid samples and glass slide drop-cast samples. The limit of detection derived from SEM characterization was estimated to be about 150 pM, comparable but slightly higher than the upper-level liquid extinction characterization for the same 80 nm AuNPs in PBS, possibly due to increased variance in nanoscale level characterization and limited sampling data.

Further, the drop-cast samples on gold surface were also analyzed by dark field scattering imaging. Here LSPR mediated scattering of incident light from AuNPs on gold surface can directly indicate the density of AuNP, based on the density of bright spots on dark field imaging. The dark field scattering images were undergoing image process to improve the contrast for spots, which were then counted using MATLAB code and averaged over 10 images (captured area 62.5 μm×62.5 μm in each image). It was observed that the density of bright spots dropped as sGP concentration increased, consistent with SEM observation. The dark field imaging method was estimated to be capable of detecting sGP with a sensitivity of about 1 nM.

Drop-Casting on Gold Film in Detecting sGP in 1×PBS with 100 nm AuNPs.

To investigate the feasibility of different AuNP sizes on sensing, we prepared 100 nm AuNP assay samples, drop-cast them on 1 mm glass slides and gold films, and characterized by UV-visible spectrometer, SEM, and dark field scattering imaging. The 100 nm AuNP concentration in assay colloid was 0.019 nM. The characterizations followed protocols described in main context method: UV-visible spectra, SEM imaging, and dark field scattering imaging characterizations. The measurement results in general showed comparable sensitivity (sub 1 nM) to that of 80 nm AuNPs and consistent with detection in PDMS well plate.

### Back-of-the-Envelope Estimation to Understand the Detection Limits

Our experimentally determined upper and lower limits in detection are thought to be correlated to the multivalent binding nature of the detection process. Suppliers informed that up to 120, 270, 460, and 730 streptavidin are bound on 40, 60, 80 and 100 nm diameter NPs, although the effective numbers are expected to be smaller. This large number of binding sites on the AuNPs makes them a strong multivalent binding sensor that is highly favorable to AuNP aggregation, even at low antigen concentration.

With very small amounts of antigens, the ultimate lower limit of detection occurs when NP precipitation decreases the AuNP monomers in the suspension significantly enough to produce a signal that is distinguishable from noise or fluctuation. This value is expected to be dependent on both the dynamic antigen-antibody binding process and the experimental setup. For example, assuming a very high affinity in sGP binding (ignoring dissociation) and four sGP bound to each aggregating AuNP (about 3-5 at 1 nM from TEM image) and setting 3% optical extinction change as the detection threshold to overcome signal variation, we could roughly estimate the LoD as 275×(3%/45%)×4=70 pM or 36× (3%/45%)×4=10 pM for 40 and 80 nm AuNPs, which are comparable to our experimental analysis. Similarly, the upper limit of detection could be estimated when the AuNPs are completely saturated with the analyte, i.e. 120×0.275=33 nM and 460×0.036=16 nM for 40 and 80 nm AuNPs, also comparable to but smaller than experimental values. The above back-of-the-envelope analysis is helpful to provide intuitive understanding of the measured dynamic range and detection limits, but it is also quite limited because it ignores the dynamic association and dissociation processes which are thought to be dependent on both the NP size and antigen-nanobody binding characteristics.

### Modeling to Understand Sensing Physics

### Rate Limiting Reaction Steps and Diffusion.

Using 80 nm AuNP as an example, we first attempted to identify the key rate determining step in the sensing mechanism. Each of the 80 nm AuNPs has ˜460 nanobodies on their surface and behaves as a multivalent sGP-binding pseudo-particle that diffuses and conjugates to each other via sGP-mediated bridging. This triggers formation of AuNP dimers oligomers, and eventually large clusters, which precipitate at the bottom of microcentrifuge tube as gravity gradually overtakes fluidic drag force. Therefore, the reaction determining steps during the sensing process include the antigen diffusion, AuNP diffusion, antigen-AuNP binding, AuNP clustering, and AuNP precipitation.

The diffusivities of AuNPs and antigens can be estimated from the Stokes-Einstein equation D=kT/(3πηd), where kT is the thermal energy at room temperature (˜4.1×10⁻²¹ Joule at 300K), n is the solution viscosity (˜1.7×10⁻³ N·sec/m² assuming 20% glycerol in water to estimate the buffer effect), and d is the particle diameter². The diffusivity is estimated Dₐ˜5.12×10⁻¹¹ m²/s for a 5 nm protein and D_{NP}˜3.2×10⁻¹² m²/s for an 80 nm AuNP. We can further estimate the diffusion length Lₐ, i.e. the distance for analyte to collide with AuNPs, as the smaller of the inter-protein separation Lₚ and inter-AuNP separation L_{NP}. L_{NP} was calculated in the range of 2 to 5 μm for 40 to 100 nm AuNPs used in our experiment following

$${L}_{NP} = \sqrt[3]{\frac{6 V}{π}} = \sqrt[3]{\frac{6}{π} \frac{1}{{c}_{NP} {N}_{A}}} ,$$

where V is volume estimated for each NP (assuming a sphere), c is the NP molar concentration, and N_{A} is the Avogadro number. Clearly, L_{NP} is determined by the NP concentration and thus is a constant once the assay is designed. Similarly, Lₚ depends on analyte concentration and can be calculated as ˜2 μm at a low sGP concentration (<100 pM) but <100 nm at a higher concentration (>1 μM). Therefore, Lₐ is mainly determined by the protein concentration, and the diffusion time tₐ˜Lₐ²/Dₐ is found to be only 0.1 to 0.2 sec, much shorter than the experimentally determined incubation assay time (3 to 7 hours).

It is important to compare here with conventional surface-incubation based assays, such as ELISA and SPR, where according to the non-slip boundary condition the surface velocity is close to zero. Differently, in our case the AuNP continues to diffuse, and its thermal velocity can be estimated by v_{NP}=KT/m_{NP}. Because of its small mass (m_{NP}=5.18×10⁻¹⁵ g for an 80 nm AuNP), there is a significantly large thermal velocity for the AuNPs v_{NP}˜0.028 m/sec. Given this velocity and the small Lₐ, we can understand that in fact the AuNPs should be constantly colliding with antigens and other nanoparticles, promoting effective mixing and antigen binding. Therefore, the diffusion process will not be a rate limiting step here, although they could significantly limit the assay time of ELISA and SPR. In another word, the antigen binding process behaves totally differently from that on an infinitely large surface in ELISA and SPR, and the measured kₒₙ, k_{off} from ELISA is limited to the surface bound molecular interactions and cannot fully predict what happens at the nanometer scale in our case. Such phenomena have been observed that the binding kinetics in solution could be significantly different from that on surface, which is attributed to mass transport and other factors. On the other hand, given the high binding affinity of analyte-ligand complex in this proposed work, we can reasonably hypothesize that the association process of this complex is fast (e.g., G protein binds to GPCR receptors within ˜0.3 sec), thus also unlikely the limiting step.

### Simplified Mathematical Modeling of AuNP Aggregation.

Here we adapted Smoluchowski's coagulation equation and modified the equation to describe our reversible AuNP aggregation process, using sGP sensing as an example. The modified equation is:

$$\frac{d {n}_{i}}{dt} = \frac{1}{2} {\sum }_{j = 1}^{i - 1} {k}_{i - j , j} {n}_{i - j} {n}_{j} - {\sum }_{j = 1}^{∞} {k}_{i , j} {n}_{i} {n}_{j} + {k}_{off} {n}_{i + 1} - {k}_{off} {n}_{i} \qquad ( 1 )$$

Where nᵢ(t) is the concentration of aggregates consisting of i AuNPs, k_{i,j} is the coagulation kernel for the aggregation of clusters consisting of i AuNPs and j AuNPs, k_{off} is the dissociation constant of sGP49-sGP conjugation (k_{off}=1.88×10⁻⁴ s⁻¹) derived from ELISA measurement. According to Brownian diffusion theory, the coagulation kernel k_{i,j} is described as:

$${k}_{i , j} = \frac{2}{3} P \frac{{k}_{B} T}{η} {( i j )}^{γ} ( {m}_{i}^{\frac{1}{{d}_{f}}} + {m}_{j}^{\frac{1}{{d}_{f}}} ) ( {m}_{i}^{- \frac{1}{{d}_{f}}} + {m}_{j}^{- \frac{1}{{d}_{f}}} ) \qquad ( 2 )$$

Where P is the probability of aggregation per collision, k_{B} is Boltzmann constant, T is temperature, η is dynamic viscosity of colloid buffer (˜1.7×10⁻³ N·s/m² for 20% glycerol in water), i and j are numbers of AuNP in each cluster, mᵢ and mⱼ are mass of each clusters, dr is the fractal dimension (˜2.1 for a typical densely aggregated cluster). Here P could be estimated as 0.0165 if using the ELISA-determined kinetic parameter kₒₙ=4.07×10⁴ M⁻¹ s⁻¹. This value is found a serious underestimate because the mass transport and antigen binding process in in-solution Nano2RED assay are significantly more effective than that for ELISA, as discussed in further herein. Indeed, we found using such a small P value could not accurately predict the assay time observed in Nano2RED. Instead, we used P=1, meaning every collision of sGP and sGP49-functionalized AuNPs will result in antigen binding. Such an assumption led to good agreement between theory and experimental observations. In fact, such a high binding efficiency is reasonable given the multivalence nature of AuNP sensors. The multivalence effectively creates a much higher “functional affinity” compared to the intrinsic affinity by monovalent binding, and could yield virtually irreversible binding process.

In equation 1, the first two terms in the right side directly come from Smoluchowski's equation that describe the AuNP aggregation process. The other two terms on the right side are added terms to describe the reversible dissociation of AuNP aggregates. For simplification, we considered only the dissociation of a cluster with N AuNPs to form a cluster of N−1 AuNPs and a monomer released back to colloid (i.e. N→N−1,1). Although the breakdown of clusters to clusters with other arbitrary numbers of AuNPs is possible (N→N−i, i), such breakdown requires multivalent sGP-sGP49 dissociation, hence the effective dissociation rate is likely to be much smaller. Moreover, a further simplification of the model by considering only the low-order oligomers and monomers interactions is justified by the fact that the concentration of nᵢ with higher i numbers (higher order) is small due to precipitation. Therefore, we could calculate the AuNP monomer concentration based on the simplified equation set and thus estimate the extinction signals by considering only the low-order oligomer-monomer interactions. This assumption is especially valid at the beginning when the assay is mixed with sGP protein, where the concentration of higher order oligomers and large clusters is near 0. Our simplified model incorporates the evolution of large clusters formation, and the equation sets are shown below:

$$\frac{d {n}_{1}}{dt} = - 2 {k}_{11} {n}_{1}^{2} - {\sum }_{i = 2}^{∞} {k}_{1 i} {n}_{i} {n}_{1} + 2 {k}_{off} {n}_{2} + {\sum }_{i = 3}^{∞} {k}_{off} {n}_{i} \qquad ( 3 )$$

$$\frac{d {n}_{2}}{dt} = 2 {k}_{11} {n}_{1}^{2} - {k}_{12} {n}_{1} {n}_{2} + {k}_{off} {n}_{3} - 2 {k}_{off} {n}_{2} \qquad ( 4 )$$

$$\frac{d {n}_{3}}{dt} = {k}_{12} {n}_{1} {n}_{2} - {k}_{13} {n}_{1} {n}_{3} + {k}_{off} {n}_{4} - {k}_{off} {n}_{3} \qquad ( 5 )$$

$$…$$

$$\frac{d {n}_{i}}{dt} = {k}_{1 i - 1} {n}_{1} {n}_{i - 1} - {k}_{1 i} {n}_{1} {n}_{i} + {k}_{off} {n}_{i + 1} - {k}_{off} {n}_{i} \qquad ( 6 )$$

By solving the equations above, we obtained the time-dependent monomer concentration, assuming 0.036 nM AuNP colloid in detecting 10 nM sGP, and further converted the concentration to optical extinction. Intuitively, the solver of this equation set showed that the monomer concentration versus time is quasi-exponential. The aggregation time constant τ_{agg}, defined as time required for concentration of AuNP monomer to drop to

$${c}_{equillibrium} + \frac{1}{e} ( {c}_{0} - {c}_{equillibrium} ) ,$$

is 0.87 hour.

Further, we calculated the monomer concentration versus time for 1.8 nM 80 nm AuNP assay in detecting 10 nM sGP. In this case, τ_{agg} is significantly shortened to 0.024 hour, or ˜36 times smaller compared to τ_{agg} using 0.036 nM AuNPs.

### Sedimentation Time.

As AuNPs cluster, the gravitational force overcomes fluidic drag, and large clusters precipitate to form sedimentation and continuously deplete AuNPs and sGP proteins in the colloid until reaching equilibrium. The sedimentation time of this progression can be estimated from the solution of the Mason-Weaver equation by τ_{sed}=z/(s·g) where z is the precipitation path (the height of colloid liquid), g is the gravitation

$$s = \frac{{d}^{2}}{18 η} ( {ρ}_{M} - {ρ}_{w} )$$

constant, s is the sedimentation coefficient (d is the aggregate diameter, n is the dynamic viscosity of the colloid buffer, ρ_{M} and ρ_{w} are the density of aggregate and colloid buffer, respectively). Clearly, the large density contrast between gold (19.3 g/cm³) and buffer (˜1 g/cm³) is also beneficial to improve the sedimentation coefficient s. Given z˜3.5 mm for 16 μL liquid in a microcentrifuge tube, we calculated that τ_{sed} decreases from 26.0 hours for an 80 nm AuNP monomer to 1.0 and 0.3 hours for 400 nm and 800 nm diameter clusters, respectively.

### Rapid Detection

### Impact of Incubation Time.

The optical images of the microcentrifuge tubes indicated the color contrast was high enough to be immediately resolved by the naked eye after vortex mixing. We have analyzed the peak extinction of the assay upper-level liquid at different incubation times, and the extinction was found to be 0.145 right after vortex, completely distinguishable from the NC sample (0.536), and it gradually decreased to 0.110 as incubation time was extended to 20 min. The analysis show that further incubation could be used as an option to moderately improve the signal contrast, but could possibly be skipped when testing speed is extremely important.

In addition, the rapid sGP detection scheme was found to be reproducible in fetal bovine serum (5% FBS), and it produced similar results in 1×PBS buffer. In both cases, sGP>1 nM can be accurately read out by the naked eye either in tubes or in a PDMS well plate.

### Portable UV-Visible Spectrometer as Readout.

The portable UV-visible spectrometer system consists of a smartphone sized Ocean Optics UV-visible spectrometer (8.8×6.3×3.1 cm³), a lamp source module (15.8×13.5×13.5 cm³), alignment clamps, and an electronic recording device (such as a laptop or a smart phone). Here, 80 nm AuNP colloid (30 μL) is mixed with sGP testing buffer (10 μL 5% FBS), vortexed and centrifuged at 1,200×g (3,500 rpm) for 1 min. After 20 minutes of incubation, the assay colloids were vortexed for 15 seconds and the upper-level liquid were loaded into 4 mm-diameter wells on a 3 mm thick PDMS plate. The light from the lamp transmitted through the colloid, whereas the rest area of the diced fused silica was covered in black to block stray light transmission. Transmitted light was collected by the spectrometer through a fiber waveguide. The extinction spectra, measured by portable spectrometer (Ocean Optics), were in general highly consistent with that measured by microscope-coupled spectrometer (Horiba iHR320), with a slightly increased signal noise. The extinction peak values at the resonance wavelength (˜559 nm) of the two measurements were in high agreement, with a small difference within 15.8%. This could possibly be attributed to different signal collection setup (10× objective with N_{A} of 0.3 in lab-based spectrometer versus waveguide collecting a nearly collimated beam in portable spectrometer). The optical signals measured by portable spectrometer were able to distinguish sGP at 100 pM (E_{100 pM}=0.509) from the reference (E_{NC}=0.542, no sGP), with a dynamic range (10 pM to 1 μM) and limit of detection (˜42 pM) comparable to that of the rapid detection of sGP in serum and 1×PBS.

Rapid sGP Detection with a Portable, Electronic Readout Device

Extinction spectrometric analysis provides quantitative and accurate diagnostics but requires bulky spectrometer systems that are more suitable for lab use. We demonstrated the feasibility of detecting sGP in FBS using a cost-efficient, portable UV-visible spectrometer system for field deployment. Additionally, we developed a homemade LED-photodiode based electronic readout system with significantly reduced system cost to deliver accurate and sensitive detection results comparable to a lab-based spectrometer system. Here, an LED light source emitted narrowband light at the AuNP extinction peak (λ_{P}=560 nm, FWHM_{P}=40 nm), which transmitted through the upper-level assay liquid and then was collected by a photodiode. As a result, a photocurrent or photovoltage was generated on a serially connected load resistor in a simple circuitry that can be easily integrated and scalably produced. In practice, we 3D-printed a black holder to snug-fit a microcentrifuge tube, and mounted the LED and photodetector on two sides of the holder. The LED and photodiode were powered by alkaline batteries (3V and 4.5V, respectively), and the bias voltages were set to ensure wide-range detection of sGP proteins without saturating the photodetectors. Using 80 nm sGP49-functionalized AuNP Nano2RED assays, sGP was detected in diluted FBS (5%) by reading the photovoltage signals with a handheld multimeter. Compared to lab-based spectrometric readout (in blue), the electronic readout displayed identical dynamic range, but slightly improved LOD (27 pM compared to 80 pM).

Nano2RED with Co-Binders and Testing in Different Biological Buffers

### The Impact of Biological Buffer Concentration.

Using FBS as a buffer and sGP49 functionalized 80 nm AuNPs for sensors, we have investigated the effect of buffer concentration. We found that high concentration FBS (50%) produced smaller optical contrast for readout compared to low concentration, <20% FBS. Further, 5% FBS displayed more consistent results and a slightly larger dynamic range for detection. For consistency in comparison, we chose 5% as the concentration of all biological buffers to be tested in the co-binder experiments.

sGP Detection Using Co-Binder sGP49/sGP7.

Two different biotinylated nanobodies, sGP49 and sGP7, were surface functionalized to streptavidin coated AuNP similar to the method described earlier for creating two different sets of functionalized AuNP colloidal solutions. The concentration of functionalized AuNP colloids were re-adjusted to get an optimal extinction level. sGP stock solution underwent serial dilution to create an sGP analyte solution with concentrations of 4 μM to 400 fM in selected detection media. The final composition of PBS detection media was composed of 1×PBS, 20% v/v glycerol and 1 wt % BSA while that of FBS, HPS (Human pooled serum), and WB (Whole blood) detection media had a final concentration of 1×PBS, 20% v/v glycerol and 1 wt % BSA and 20% of either FBS, HPS, or WB which resulted in a final concentration of FBS, HPS, or WB in the detection assay to be 5%. Solutions of sGP49-functionalized AuNPs, sGP7-functionalized AuNP, and sGP were mixed in a 500 μL Eppendorf tube at a ratio of 3:3:2 and thoroughly vortexed. After mixing, the detection assay was centrifuged (accuSpin Micro 17, Thermo Fisher) at 3,500 rpm (1,200×g) for 1 minute. AuNPs were highly concentrated at the bottom of Eppendorf tube. After 20 minutes of incubation, the colloidal assay was vortexed (mini vortexer, Thermo Fisher) at 800 rpm for 15 seconds to thoroughly remix free AuNP monomers into the colloid. Following this, spectrometric and electronic characterizations were done in a way similar to that described previously.

### RBD Detection Using Co-Binder RBD8/RBD10.

Two different biotinylated nanobodies, RBD10 and RBD8, were surface functionalized to streptavidin coated AuNP similar to the method described earlier for creating two different sets of functionalized AuNP colloidal solutions. The concentration of functionalized AuNP colloids were re-adjusted to get an optimal extinction level. RBD stock solution underwent serial dilution to create an RBD analyte solution with concentrations of 4 μM to 4 pM in selected detection media. The final composition of PBS detection media was composed of 1×PBS, 20% v/v glycerol and 1 wt % BSA while that of FBS, HPS (Human pooled serum), and WB (Whole blood) detection media had a final concentration of 1×PBS, 20% v/v glycerol and 1 wt % BSA and 20% of either FBS, or HPS which resulted in a final concentration of FBS or HPS in the detection assay to be 5%. Solutions of RBD8-functionalized AuNPs, RBD10-functionalized AuNP, and RBD proteins were mixed in a 500 μL Eppendorf tube at a ratio of 3:3:2 and thoroughly vortexed at 800 rpm for 15-20 seconds. After mixing, the detection assay was centrifuged (accuSpin Micro 17, Thermo Fisher) at 3,500 rpm (1,200×g) for 1 minute. After 20 minutes of incubation, the colloidal assay was vortexed (mini vortexer, Thermo Fisher) at 800 rpm for 15 seconds. Then spectrometric and electronic characterizations were performed.

### Detection Error and LOD in Phage ELISA.

To determine the Phage ELISA LOD analysis similar to the method described in previous section was performed (Table 2). For consistency in LOD analysis, we analyzed the results with three different methods for sigma estimation. The first method is the traditional way of determining LOD, which only takes into account the standard deviation of blank (or NC) sample measurement (termed σ_{NC}). The two additional methods, i.e., Pooled Sigma All (σ_{PSA}) and Pooled Sigma 4 (σ_{PS4}) employ a pooled variance method which is used for statistical analysis of different populations but with similar variances to give a more robust and consistent result. While σ_{PSA} takes into account all of the samples, σ_{PS4} takes onto account only the last four samples.

Here for ELISA, the measurement noise is strongly correlated to signal level and sample concentration (Table 2). As a result, σ_{PSA} is quite high due to the consideration of high-concentration samples. Yet the use of σ_{NC} makes the result very susceptible to experimental errors in measuring the NC sample, which could seriously affect the LOD by orders of magnitude given a small signal difference at low sample concentration. Comparatively, σ_{PS4} provides a more balanced estimation for reagent characterization, because the last 4 sample data (including the NC sample) have similar signal and noise levels and the average in fact provides a more consistent estimation of experimental errors.

### Detection Error and LOD in Rapid Test.

For consistent data acquisition, it was imperative that the spectrometric data acquisition be done as soon as possible in order to minimize error introduced by gravitational precipitation of AuNP. Here it was noticed that the traditional method of determining LOD, i.e. σ_{NC}, negatively affected the consistency in LOD determination (Table 3). This can be attributed to the nature of data collection in that optical focusing varies from one measurement to the next. In comparison, σ_{PSA} and σ_{PS4} provide better consistency and robustness in determining the standard deviation required for LOD measurement. This is particularly true when the error measured from the NC sample is too high or too low compared to the average errors measured across the whole sample set. Here, we choose the σ_{PSA} method for further analysis of the data because of its better data consistency. Table 2 shows the summary of the analysis.

TABLE 3
LOD analysis of Ebola sGP and COVID RBD detection by Nano2RED

| | | Readout | σ (A.U.) | | | LOD (pM) | | |
|---|---|---|---|---|---|---|---|---|
| Antigen | Media | method | σ_{PS4} | σ_{PSA} | σ_{NC} | σ_{PS4} | σ_{PSA} | σ_{NC} |
| Ebola | PBS | Spectrometric | 0.005211 | 0.005747 | 0.0042 | 0.133 | 0.161 | 0.0913 |
| sGP | FBS | | 0.007094 | 0.005944 | 0.00914 | 1.65 | 1.05 | 3.75 |
| | HPS | | 0.004836 | 0.00548 | 0.00558 | 1.09 | 1.26 | 1.29 |
| | HPS | Electronic | 0.004459 | 0.004518 | 0.00483 | 0.134 | 0.135 | 0.142 |
| | WB | Spectrometric | 0.005841 | 0.005276 | 0.00701 | 18.24 | 16.9 | 21.44 |
| COVID- | PBS | Spectrometric | 0.005325 | 0.00527 | 0.00612 | 1.51 | 1.43 | 3.4 |
| 19 RBD | FBS | | 0.005643 | 0.005621 | 0.00522 | 5.28 | 5.23 | 4.39 |
| | HPS | | 0.00474 | 0.0046 | 0.00346 | 32.93 | 22.25 | 7.29 |
| | WB | | 0.00608 | 0.0087 | 0.00671 | 121.91 | 253.3 | 153.65 |
| | HPS | Electronic | 0.00419 | 0.004288 | 0.00311 | 1.34 | 1.35 | 1.15 |

### Detection of sGP and RBD in Serum and Blood

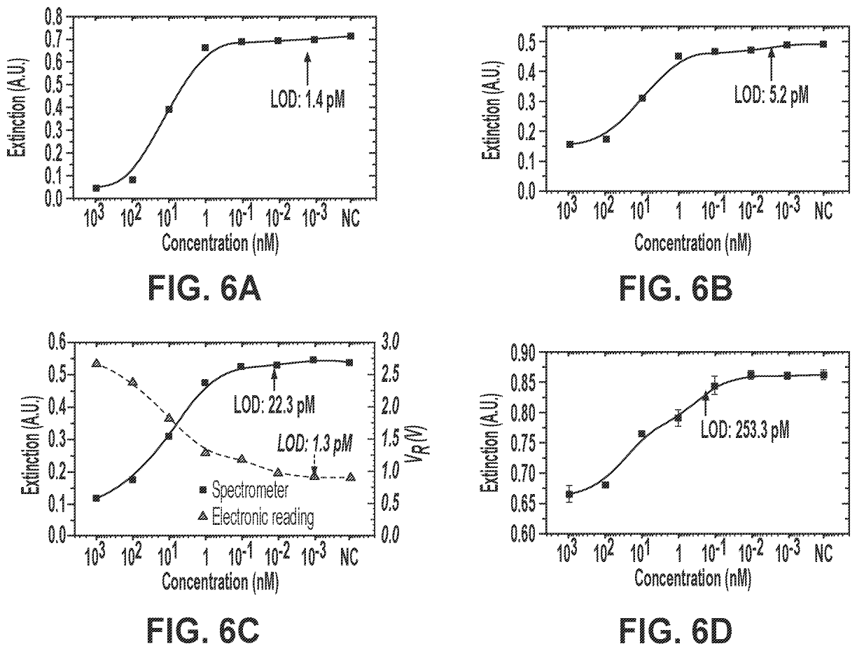

We further evaluated the use of co-binding nanobodies in sGP and RBD sensing (FIG. 6), i.e., sGP49/sGP7 for sGP and RBD8/RBD10 for RBD (FIG. 4), and performed Nano2RED tests in different buffers, including PBS, FBS, human pooled serum (HPS), and whole blood (WB) (additional data is described herein). The incubation and rapid assay formats with the assay performance, instrument costs, and LODs were summarized in Tables 1 and 4, and additional data on measurement variance (sigma) and LOD were summarized in Tables 2 and 3. There are several notable observations. First, using sGP sensing as an example and comparing to previously reported results (Table 3), Nano2RED with spectrometric and electronic readout consistently produced ˜130 fM to 1.3 pM LOD (or ˜10 to ˜100 μg/mL) in PBS, FBS, and HPS. It is noted that a very recently reported co-binder-based D4 assay format reported ˜30 μg/mL LOD in human serum, and was able to detect the Ebola virus earlier than PCR in a monkey model. In LOD comparison, the sensitivity of Nano2RED (˜10 μg/mL with electronic readout) is even better, indicating its competitiveness in high-precision diagnostics.

TABLE 4
Performance of Nano2RED in SARS-CoV-2 RBD protein sensing

| | | | Readout | Readout | | | | Diagnostic | |
|---|---|---|---|---|---|---|---|---|---|
| Assay | Readout | Readout | system | system | | Assay | | sensitivity | |
| format | method | system | cost | size | | time | Buffer | pM | pg/mL |
| Centrifuge- | Colorimetric | Eyes or smart | ~$200 | ~78 | cm³ | 5-20 | FBS | 1000 | 29180 |
| accelerated | | phones | (phone) | | | min | | | |
| (rapid) | Spectrometric | Lab | $25,000 | ~3 | m³ | | PBS | 1.4 | 42 |
| | | microscope | | | | | FBS | 5.23 | 153 |
| | | | | | | | HPS | 22.3 | 649 |
| | | | | | | | WB | 253.3 | 7372 |

| | | | | | | |
|---|---|---|---|---|---|---|
| Electronic | LED & | ~$5 | 1.1 × 1.1 × | HPS | 1.35 | 39 |
| | photodetector | | 3.3 cm³ | | | |

Additionally, our study (Table 1) also revealed the importance of a systematic assay design strategy, from molecular binding to signal transduction and readout, to optimize antigen detection. It is clear that the co-binder pair improved the LOD by 10 to 100 times compared to the mono-binder (sGP49, k_{D} 4.6 nM), despite a relatively low k_{D} of 199 nM for the second binder (sGP7) (FIG. 4). This improved sensitivity is likely because the co-binders have a favorable, non-competitive binding configuration that serves to improve antigen binding and AuNP aggregation. Uniquely, the use of a portable and inexpensive electronic readout did not negatively affect the LOD of Nano2RED, but rather improved it compared to spectrometric readout (FIG. 6i, and Table 1 and 4). This can be attributed to smaller 3-sigma errors in the electronic readout (Table 3), partly due to a larger signal fluctuation in optical imaging caused by manual operation, such as in focusing. Here, the electronic signal is mainly dependent on the circuit elements but much less dependent on operators' judgement, and thus potentially more reliable and accurate. In addition, the use of biological buffers could also affect detection. For both Ebola sGP and SARS-CoV-2 RBD, the LOD increased by about 5-10 times in serum (FBS and HPS) than in PBS, and further increased by another 10 times in WB. Additionally, the colorimetric readout by the naked eye was capable of detecting both antigens in serum at concentrations higher than 100 pM or 1 nM (FIG. 8); however, it became challenging to do so in WB, mainly due to the fact that WB absorbs in short wavelengths and causes background color interference with AuNPs. However, the spectrometric readout could still readily identify the sGP or RBD extinction signals from the background WB absorption for accurate detection, indicating the feasibility of Nano2RED for field use with minimized sample preparation.

Fundamentally different from conventional high-sensitivity antigen diagnostics that usually require bulky and expensive readout systems, as well as long assay time, Nano2RED is an affordable and accessible diagnostic technology. For example, ultrasensitive sGP sensing using NP-enhanced fluorescent readout would require 3-4 hours of image processing to reduce noise for optimal sensing, and these fluorescent systems usually require cubic meter space and cost $40,000 or more (a high-end fluorescent camera with high signal-to-noise ratio is ˜$25,000). Similarly, a D4 co-binder assay requires a lab-based bulky fluorescent system and ˜60 min assay time to achieve PCR-comparable diagnostic sensitivity. Its sensing performance drops ˜10 times to 100 μg/mL when using a customized fluorescent system, which costs ˜$1,000 and occupies ˜3,000 cm³. The performance further decreases to 6,000 μg/mL when using LFA with colorimetric readout. Clearly standing apart from the rest, Nano2RED utilizes miniaturized and low-cost semiconductor devices for signal readout rather than a fluorescent system. Therefore, it has a very small footprint (4 cm³ for tube holder, or <100 cm³ for the whole system, including batteries and meters, which all could be miniaturized on a compact circuit board in the future), is very low cost (LED and photodiode each <$1 here, but can be <$0.1 when used at large scale, with the total system cost estimated well below $5), and offers a rapid readout (5 to 20 min, depending on incubation time after centrifugation). Further, the electronic readout is more accurate than the colorimetric readout, more accessible, without color vision limitations, and more readily available for data storage in computers or online databases for real-time or retrospective data analysis. Additionally, we have estimated the reagent cost in Nano2RED is only about $0.01 per test (Supplementary section 9), since it requires only a small volume (˜20 μL) of reagents.

We tested sGP against GP1,2, a homotrimer glycoprotein transcribed from the same GP gene and sharing its first 295 residues with sGP, both in FBS. The majority of GP1,2 can be found on virus membranes whereas a small portion is released into the patient's bloodstream. The close relevance of GP1,2 to sGP makes it a very strong control molecule to assess our assay's specificity. Indeed, GP1,2 did not produce detectable signals unless higher than 1 nM, indicating a high selectivity over a broad concentration range (100 fM to 1 nM, or 4 logs) where minimal nanobody binding or AuNP aggregation occurred. A high assay specificity is crucial for minimizing false positive diagnosis of infectious diseases, which could lead to unnecessary hospitalizations and even infections. Considering that 10 nM and higher sGP concentration is typical for EVD patients, Nano2RED is particularly suitable for high-speed mass screening of EVD susceptible populations. Further, SARS-CoV-2 RBD proteins were also detected in the single-digit picomolar range in PBS, FBS, and HPS, with the best LOD (Table 3, 1.3 pM, or ˜40 μg/mL) again achieved with electronic readout. The LOD in RBD sensing is ˜10 times higher compared to sGP sensing, mainly attributed to lower binding affinities of the nanobodies obtained from a single-round biopanning (FIG. 4). Tighter binders can be selected using more biopanning rounds; however, the detection of RBD, a monomeric protein target, serves to demonstrate the general feasibility of the Nano2RED co-binding assay in detection of a broad range of antigens, regardless of their complex molecular structures. Considering the fact that the spike protein is a trimer and each SARS-CoV-2 particle is covered with ˜20 copies of such trimers, the detection of SARS-CoV-2 virus particles could behave differently. The detection of SARS-CoV-2 particles might have a better sensitivity. The detection of viral particles from patient samples would itself be quite exciting future studies but beyond the scope of this work. Nevertheless, the broadband dynamic range (˜100 fM to 1 μM for co-binders, or 7 logs), high sensitivity, high specificity, and broad applications therefore make our Nano2RED assay highly feasible for precise antigen quantification and detection of early-stage infection.

### CONCLUSIONS AND OUTLOOK

We have demonstrated a generalizable and rapid assay design and pipeline that combines fast affinity reagent selection and production with nanometer-scale theoretical analysis and experimental characterization for optimized sensing performance. Synthetic, high-affinity, co-binding nanobodies, which could be quickly produced by a phage display selection method from a premade combinatorial library for any given antigen, proved to be effective in detecting dimeric Ebola sGP and monomeric SARS-CoV-2 RBD proteins. The Nano2RED utilized unique signal transduction pathways to convert biological binding into electronic readout. Using simple electronic circuitry, it starts with AuNP aggregation (governed by dynamic antigen-nanobody interactions described by Langmuir isotherm), which triggers AuNP cluster sedimentation (explained by Mason-Weaver equation), and then enables AuNP-concentration-dependent optical extinction (following Beer-Lambert law). The use of AuNPs for in-solution testing serves to greatly facilitate fluidic transport and antigen binding at nanometer scale. Nano2RED eliminates the need for long-time incubation due to slow analyte diffusion in conventional ELISA and other plane surface-based assays, as well as its associated cumbersome washing steps. The introduction of brief centrifugation and vortex mixing further greatly shortens the aggregation and sedimentation time, enabling rapid tests (within 5 to 20 min) without sacrificing sensitivity or specificity. Our data showed that Nano2RED is highly sensitive (sub-picomolar or ˜10 μg/mL level for sGP) and specific in biological buffers while also affordable and accessible. Importantly, the portable electronic readout, despite being very simple and inexpensive (<$5), proved to be more reliable and sensitive than colorimetric and even spectrometric readout. Nano2RED can be applied for lab tests to detect early-stage virus infection at a high sensitivity potentially comparable to PCR. It can also be used for high-frequency at-home or in-clinic diagnostics, as well as in resource-limited regions, which could greatly enhance control of disease transmission. The digital data format will also reduce human intervention in data compiling and reporting, while facilitating fast and accessible data analysis. Nano2RED may find immediate use in the current COVID-19 pandemic for both antigen and antibody detection, as well as preparing for future unforeseeable new outbreaks.

### Methods

Materials. Phosphate-buffered saline (PBS) was purchased from Fisher Scientific. Bovine serum albumin (BSA) and molecular biology grade glycerol were purchased from Sigma-Aldrich. Fetal bovine serum (FBS) was purchased from Gibco, Fisher Scientific. FBS was used without heat inactivation to best reflect the state of serum collected in field. Polyvinyl alcohol (PVA, Mw 9,000-10,000) was purchased from Sigma-Aldrich. Sylgard 184 silicone elastomer kit was purchased from Dow Chemical. DNase/RNase-free distilled water used in experiments was purchased from Fisher Scientific. Phosphate Buffered Saline with Tween 20 (PBST), Nunc MaxiSorp 96 well ELISA plate, streptavidin, 1% casein, 1-Step Ultra TMB ELISA substrate solution, and isopropyl-β-D-galactopyranoside (IPTG) were purchased from Thermo Fisher Scientific. HRP-M13 major coat protein antibody was purchased from Santa Cruz Biotechnology. Sucrose and imidazole were purchased from Sigma-Aldrich. A 5 mL HisTrap column, HiLoad 16/600 Superdex 200 pg column, and HiPrep 26/10 desalting column were purchased from GE Healthcare. BirA-500 kit was purchased from Avidity. Streptavidin (SA) Biosensors were purchased from ForteBio. The streptavidin functionalized AuNPs, dispersed in 20% v/v glycerol and 1 wt % BSA buffer, were purchased from Cytodignostics. Thiolated carboxyl polyethylene glycol linker was self-assembled on AuNP through a thiol-sulfide reaction. Streptavidin was then surface functioned through amine-carboxyl coupling by N-Hydroxysuccinimide/1-Ethyl-3-(3-dimethylaminopropyl) carbodiimide (NHS/EDC) chemistry.

Phage display selection. sGP and RBD (GenScript) protein binder selection was done according to previously established protocols. In brief, screening was performed using biotin and biotinylated target protein-bound streptavidin magnetic beads for negative and positive selections, respectively. Prior to each round, the phage-displayed nanobody library was incubated with the biotin-bound beads for 1 h at room temperature to remove off-target binders. Subsequently, the supernatant was collected and incubated with biotinylated-target protein-bound beads for 1 h. Beads were washed with 10×0.05% PBST (1×PBS with 0.05% v/v Tween 20) and phage particles were eluted with 100 mM triethylamine. A total of three rounds of biopanning were performed with decreasing amounts of antigen (200 nM, 100 nM, 20 nM). Single colonies were picked and validated by phage ELISA followed by DNA sequencing.

Single phage ELISA. ELISAs were performed according to standard protocols. Briefly, 96 well ELISA plates (Nunc MaxiSorp, Thermo Fisher Scientific) were coated with 100 μL 5 μg/mL streptavidin in coating buffer (100 mM carbonate buffer, pH 8.6) at 4° C. overnight. After washing with 3×0.05% PBST (1×PBS with 0.05% v/v Tween 20), each well was added to 100 μL 200 nM biotinlyated target protein and incubated at room temperature for 1 h. Each well was washed by 5×0.05% PBST, blocked by 1% casein in 1×PBS, and added to 100 μL single phage supernatants. After 1 h, wells were washed by 10×0.05% PBST, added to 100 μL HRP-M13 major coat protein antibody (RL-ph1, Santa Cruz Biotechnology; 1:10,000 dilution with 1×PBS with 1% casein), and incubated at room temperature for 1 h. A colorimetric detection was performed using a 1-Step Ultra TMB ELISA substrate solution (Thermo Fisher Scientific) and OD450 was measured with a SpectraMax Plus 384 microplate reader (Molecular Devices).

Mono-binders purification and biotinlyation. sGP7, sGP49, RBD8, and RBD10 mono-binders were expressed as a C-terminal Avi-tagged and His-tagged form in E. coli and purified by Ni-affinity and size-exclusion chromatography. In brief, E. coli strain WK6 was transformed and grown in TB medium at 37° C. to an OD600 of ˜0.7, then induced with 1 mM isopropyl-β-D-galactopyranoside (IPTG) at 28° C. for overnight. Cell pellets were resuspended in 15 mL ice-cold TES buffer (0.2 M Tris-HCl pH 8.0, 0.5 mM EDTA, 0.5 M sucrose) and incubated with gently shaking on ice for 1 h, then added to 30 mL of TES/4 buffer (1:4 dilution of the TES buffer in ddH₂O) and gently shaken on ice for 45 min. Cell debris was removed by centrifugation at 15,000×g, 4° C. for 30 mins. The supernatant was loaded onto a 5 mL HisTrap column (GE Healthcare) pre-equilibrated with the lysis buffer (50 mM sodium phosphate, pH 8.0, 300 mM NaCl, 10 mM imidazole, 10% glycerol). The column was washed with a washing buffer (50 mM sodium phosphate, pH 8.0, 300 mM NaCl, 20 mM imidazole, 10% glycerol) and then His-tagged proteins were eluted with an elution buffer (50 mM sodium phosphate, pH 8.0, 300 mM NaCl, 250 mM imidazole, 10% glycerol). Eluates were loaded onto a HiLoad 16/600 Superdex 200 pg column (GE Healthcare) pre-equilibrated with a storage buffer (1×PBS, 5% glycerol). Eluted proteins were concentrated, examined by SDS-PAGE, and quantified by a Bradford assay (BioRad), then flash frozen in 100 μL aliquots by liquid N₂ and stored at −80° C.

The purified protein was biotinylated by BirA using a BirA-500 kit (Avidity). Typically, 100 μL BiomixA, 100 μL BiomixB, and 4 μL 1 mg/mL BirA were added to 500 μL protein (˜1 mg/mL), and adjusted to a final volume of 1000 μL with nuclease-free water (Ambion). The biotinylation mixture was incubated at room temperature for 1 h and then loaded onto a HiPrep 26/10 desalting column (GE Healthcare) pre-equilibrated with a storage buffer (1×PBS, 5% glycerol) to remove the free biotin.

Binding kinetics analysis. The four mono-binders' binding kinetics were analyzed using an Octet RED96 system (ForteBio) and Streptavidin (SA) biosensors. 200 nM biotinylated sGP or RBD target protein was immobilized on SA biosensors with a binding assay buffer (1×PBS, pH 7.4, 0.05% Tween 20, 0.2% BSA). Serial dilutions of mono-binder were used for the binding assay. Dissociation constants (K_{D}) and kinetic parameters (kₒₙ and k_{off}) were calculated based on global fit using Octet data analysis software 9.0. For co-binder validation, sGP or RBD bound SA biosensors were first dipped into the sGP49 or RBD10 wells 750 s for saturation, then incubated with sGP7 or RBD8 for 750 s.

Determination of detection sensitivity using a co-binder sandwich ELISA assay. In order to validate and determine the detection sensitivity of co-binders, a sandwich ELISA-like assay was performed. Briefly, 96 well ELISA plates (Nunc MaxiSorp, Thermo Fisher Scientific) were coated with 100 μL 5 μg/mL streptavidin in coating buffer (100 mM carbonate buffer, pH 8.6) at 4° C. overnight. After washing with 3×0.05% PBST (1×PBS with 0.05% v/v Tween 20), each well was added to 100 μL 200 nM biotinlyated sGP49 or RBD10 (˜100 nM) protein and incubated at room temperature for 1 h, then washed with 5×0.05% PBST and added to 100 μL serial dilutions (0 to 500 nM) of sGP or RBD protein and incubated at room temperature for 1 h. Each well was subsequently blocked by 1% casein in 1×PBS for 1 h, then added to 100 μL sGP7 or RBD8 phage supernatants. After 1 h, wells were washed by 10×0.05% PBST, added to 100 μL HRP-M13 major coat protein antibody (RL-ph1, Santa Cruz Biotechnology; 1:10,000 dilution with 1×PBS with 1% casein), and incubated at room temperature for 1 h. A colorimetric detection was performed using a 1-Step Ultra TMB ELISA substrate solution (Thermo Fisher Scientific) and OD450 was measured with a SpectraMax Plus 384 microplate reader (Molecular Devices). Limit of Detection (LoD) was calculated by mean blank+3×SD_{PS4}. More details on the SD selection is provided in (Tables 2 and 3).

Nanoparticle functionalization with nanobodies. The streptavidin surface functioned gold nanoparticles (typically ˜0.13 nM 80 nm AuNPs, 80 μL) were first mixed with an excessive amount of biotinylated nanobodies (about 1.2 μM, 25 μL). The mixture was then incubated for 2 hours to ensure complete streptavidin-biotin conjugation. Next, the mixture was purified by centrifuge (accuSpin Micro 17, Thermo Fisher) at 10,000 rpm for 10 min and repeated twice to remove unbounded biotinylated nanobodies. The purified AuNP colloid was measured by Nanodrop 2000 (Thermo Fisher) to determine the concentration. The concentration of AuNP in colloid was subsequently adjusted to get an optimal extinction level (e.g., empirically 0.048 nM for 80 nm AuNPs) and was aliquoted into 12 μL in a 500 μL Eppendorf tube.

Antigen detection. Target sGP or RBD stock solution (6 μM, in 1×PBS) underwent a 10-fold serial dilution to target concentrations (4 pM to 4 μM) in selected detection media. The final composition of PBS detection media was composed of 1×PBS, 20% v/v glycerol and 1 wt % BSA while that of FBS (Fetal Bovine Serum), HPS (Human Pooled Serum), and WB (Whole blood) detection media had a final concentration of 1×PBS, 20% v/v glycerol and 1 wt % BSA and 20% of either FBS, HPS, or WB, which resulted in a final concentration of FBS, HPS, or WB in the detection assay to be 5%. For example, for sGP detection with co-binders, solutions of sGP49-functionalized AuNPs, sGP7-functionalized AuNP, and sGP were mixed in a 500 μL Eppendorf tube at a ratio of 3:3:2 and thoroughly vortexed. When incubation-based detection was used, the solution was allowed to incubate (typically 3 hours) prior to readout. A similar protocol was used for RBD sensing.

Centrifuge enhanced rapid detection. The same protocols were followed in preparation of sGP49 surface functioned AuNP colloid. After mixing sGP49 surface functioned AuNP colloid with sGP solution, the AuNP assay colloid was centrifuged (accuSpin Micro 17, Thermo Fisher) at 3,500 rpm (1,200×g) for 1 minute. After optional incubation, the assay colloid was vortexed (mini vortexer, Thermo Fisher) at 800 rpm for 15 seconds prior to readout. A similar protocol was used for RBD sensing.

PDMS well plate fabrication. Sylgard 184 silicone elastomer base (consisting of dimethyl vinyl-terminated dimethyl siloxane, dimethyl vinylated, and trimethylated silica) was thoroughly mixed with the curing agent (mass ratio 10:1) for 30 minutes and placed in a vacuum container for 2 hours to remove the generated bubbles. The mixture was then poured into a flat plastic container at room temperature and incubated for one week, until the PDMS is fully cured. The PDMS membrane was then cut to rectangular shape, and 2 mm wells were drilled by punchers. To prevent non-specific bonding of proteins, the PDMS membrane was treated with PVA, adapted from methods described by Trantidou et al., Hydrophilic surface modification of PDMS for droplet microfluidics using a simple, quick, and robust method via PVA deposition. Microsystems & nanoengineering 2017, 3, 1-9. The as-prepared PDMS membrane and a diced rectangle shaped fused silica (500 μm thick) were both rinsed with isopropyl alcohol, dried in nitrogen, and treated by oxygen plasma (flow rate 2 sccm, power 75 W, 5 min). Immediately after, the two were bonded to form a PDMS well plate. The plate was further oxygen plasma treated for 5 min and immediately soaked in 1% wt. PVA in water solution for 10 min. Then, it was dried by nitrogen, heated on a 110° C. hotplate for 15 min, and cooled to room temperature by nitrogen blow.

UV-visible spectrometric measurement and dark field scattering characterizations. The UV-visible spectra and dark field imaging were performed using a customized optical system (Horiba), comprising an upright fluorescence microscope (Olympus BX53), a broadband 75 W Xenon lamp (PowerArc), an imaging spectrometer system (Horiba iHR320, spectral resolution 0.15 nm), a low-noise CCD spectrometer (Horiba Syncerity), a high-speed and low-noise EMCCD camera (Andor iXon DU897 Ultra), a vision camera, a variety of filter cubes, operation software, and a high-power computer. For spectral measurement, the PDMS plate loaded with upper-level assay samples or drop-cast samples on a glass slide was placed on the microscope sample stage. Light transmitted through PDMS well plate was collected by a 50×objective lens (NA=0.8). The focal plane was chosen at the well plate surface to display the best contrast at the hole edge. A 10×objective lens (NA=0.3) was used for drop-cast samples. The signals were typically collected from the 350 nm to 800 nm spectral range with integration time of 0.01 s and averaged 64 times. The dark field scattering was illuminated by an xeon lamp, collected by a 100×dark field lens (NA=0.9), and imaged by an EMCCD camera. The integration time was set to 50 ms. For each drop-cast sample spot, ten images were taken from different areas in the spot. The size for the area taken in each dark-field scattering image was 62.5 μm×62.5 μm.

SEM imaging of drop-cast samples on gold. The SEM image was taken by a Hitachi S4700 field emission scanning electron microscope at 5 kV acceleration voltage and magnification of 15,000. For each drop-cast sample spot, ten images were taken from different regions in the spot. The size of the region taken in each SEM image was 8.446 μm×5.913 μm.

TEM to image AuNP precipitates. The upper-level assay liquid was removed from the microcentrifuge tube until 2 to 3 μL of sample containing AuNP precipitates were left. The tube was vortexed thoroughly, and 2 μL of remaining samples were pipetted, and coated on a Cu grid (Electron Microscopy Sciences, C flat, hole size 1.2 μm, hole spacing 1.3 μm) that was pre-treated on both sides with oxygen plasma (30 seconds). The Cu grid was plunge frozen in ethane using Vitrobot plunge freezer (FEI). The blot time was set to 6 sec. After plunging, the sample was soaked in liquid nitrogen for long-term storage. FEI Tecnai F20 transmission electron microscope (200 kV accelerating voltage) was used for CryoTEM imaging. 25 high-resolution TEM images were taken for 1 μM, 1 nM sGP in PBS samples and reference samples, respectively. The size of the area taken in each image was 4.476 μm×4.476 μm.

Portable spectrometric readout. The assay colloids were initially characterized by a miniaturized portable UV-visible spectra measurement system. OSL2 fiber coupled illuminator (Thorlabs) was used as the light source. The light passed through the 4 mm diameter wells loaded with assay colloid and coupled to Flame UV-visible miniaturized spectrometer (Ocean optics) for extinction spectra measurement. The signals were averaged from six scans (each from 430 nm to 1100 nm) and integrated for 5 seconds in each scan.

Electronic readout with rapid test. A LED-photodiode electronic readout system consists of three key components: a LED light source, a photodiode, and a microcentrifuge tube holder. The centrifuge tube holder was 3D printed using ABSplus P430 thermoplastic. An 8.6 mm diameter recess was designed to snuggly fit a standard 0.5 mL Eppendorf tube. 2.8 mm diameter holes were open on two sides of the microcentrifuge tube holder to align a LED (597-3311-407NF, Dialight), the upper-level assay liquid, and a photodiode (SFH 2270R, Osram Opto Semiconductors). The LED was powered by two Duracell optimum AA batteries (3 V) through a serially connected 35Ω resistor to set the LED operating point. The photodiode was reversely biased by three Duracell optimum AA batteries (4.5 V) and serially connected to a 7 MΩ load resistor. The photocurrent that responds to intensity of light transmitted through the assay was converted to voltage through the 7 MΩ load resistor and measured with a portable multimeter (AstroAl AM33D).

Estimate of limit of detection for Nano2RED. In our work, limit of detection (LOD) was calculated according to the International Union of Pure and Applied Chemistry definition, that is, the concentration at which the measured response is able to distinguish from the reference signal by three times the standard deviation in measurements. For optical measurement, we used LoD=c(E_{NC}−3σ). Here, the reference was averaged over three measurements of the negative control (NC) sample. Whereas for electronic measurement, we used LoD=c(V_{NC}+3σ), where V_{NC} is the readout voltage for NC sample. We compared different methods to estimate σ, i.e., the conventional way considering only the NC sample (σ_{NC}), using pooled sigma from all measurements (σ_{PSA}), and pooled sigma from the four lowest concentrations (σ_{PS4}) (Table 2). We noticed in our case that using σ_{PSA} and σ_{PS4} both yielded more consistent reporting of LOD compared to using σ_{NC}, and therefore the sensor LOD was estimated with σ_{PSA} for its best consistency (Table 1). This consistency could be attributed to the nature of our data collection is a physical process and less dependent on reagent concentration compared to conventional ELISA. Particularly when using spectrometric readout, the noise is strongly affected by optical focusing and could happen to any data sets; and therefore the overall average provides a better estimate of the empirical errors. Differently, for ELISA measurement, the sigma is much smaller at lower concentrations, so we used conventional σ_{NC} for LOD determination.

Some further aspects are defined in the following clauses:

Clause 1: A method of detecting severe acute respiratory syndrome coronavirus-2 (SARS-CoV-2) in a sample, the method comprising: contacting the sample with a plurality of gold nanoparticles (AuNPs) and/or other plasmonic metal nanoparticles (MNPs) that are conjugated with at least two sets of antibodies, or antigen binding portions thereof, wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein under conditions sufficient for the first and second set of antibodies, or the antigen binding portions thereof, to bind to the first and second epitopes of the SARS-CoV-2 proteins in the sample to produce bound SARS-CoV-2 proteins; or contacting the sample with a plurality of gold nanoparticles (AuNPs) and/or other plasmonic metal nanoparticles (MNPs) that are conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins; and, detecting the SARS-CoV-2 proteins when one or more aggregations of the bound SARS-CoV-2 proteins form with one another, thereby detecting the SARS-CoV-2 in the sample.

Clause 2: The method of Clause 1, wherein the antibodies comprise monoclonal antibodies.

Clause 3: The method of Clause 1 or Clause 2, wherein the first and second set of antibodies, or the antigen binding portions thereof, comprise nanobodies.

Clause 4: The method of any one of the preceding Clauses 1-3, wherein the SARS-CoV-2 protein comprises a receptor-binding domain (RBD).

Clause 5: The method of any one of the preceding Clauses 1-4, wherein the detection step comprises determining a change in absorbance at a resonance wavelength of the AuNPs and/or other MNPs.

Clause 6: The method of any one of the preceding Clauses 1-5, comprising quantifying an amount of the SARS-CoV-2 proteins and/or the SARS-CoV-2 in the sample.

Clause 7: The method of any one of the preceding Clauses 1-6, further comprising centrifuging the aggregations of the bound SARS-CoV-2 proteins prior to and/or during the detecting step.

Clause 8: The method of any one of the preceding Clauses 1-7, further comprising freezing the aggregations of the bound SARS-CoV-2 proteins prior to the detecting step.

Clause 9: The method of any one of the preceding Clauses 1-8, comprising drop casting the aggregations of the bound SARS-CoV-2 proteins prior to the detecting step.

Clause 10: The method of any one of the preceding Clauses 1-9, comprising obtaining the sample from a subject.

Clause 11: The method of any one of the preceding Clauses 1-10, comprising administering one or more therapies to the subject when the SARS-CoV-2 is detected in the sample.

Clause 12: The method of any one of the preceding Clauses 1-11, comprising detecting the SARS-CoV-2 within about 20 minutes or less of obtaining the sample from the subject.

Clause 13: The method of any one of the preceding Clauses 1-12, comprising repeating the method using one or more longitudinal samples obtained from the subject.

Clause 14: The method of any one of the preceding Clauses 1-13, wherein the sample comprises blood, plasma, serum, saliva, sputum, or urine.

Clause 15: The method of any one of the preceding Clauses 1-14, wherein the detecting step comprises measuring a colorimetric change when the one or more aggregations of the bound SARS-CoV-2 proteins form with one another.

Clause 16: The method of any one of the preceding Clauses 1-15, comprising visually detecting the colorimetric change when the one or more aggregations of the bound SARS-CoV-2 proteins form with one another.

Clause 17: The method of any one of the preceding Clauses 1-16, comprising detecting the colorimetric change when the one or more aggregations of the bound SARS-CoV-2 proteins form with one another using a spectrometer.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, kits, reaction mixtures, devices, and/or systems or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting severe acute respiratory syndrome coronavirus-2 (SARS-CoV-2) in a sample, the method comprising:
contacting the sample with a plurality of gold nanoparticles (AuNPs) and/or other plasmonic metal nanoparticles (MNPs) that are conjugated with at least two sets of antibodies, or antigen binding portions thereof, wherein at least a first set of antibodies, or antigen binding portions thereof, binds to a first epitope of a SARS-CoV-2 protein and wherein at least a second set of antibodies, or antigen binding portions thereof, binds to a second epitope of a SARS-CoV-2 protein under conditions sufficient for the first and second set of antibodies, or the antigen binding portions thereof, to bind to the first and second epitopes of the SARS-CoV-2 proteins in the sample to produce bound SARS-CoV-2 proteins; or
contacting the sample with a plurality of gold nanoparticles (AuNPs) and/or other plasmonic metal nanoparticles (MNPs) that are conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins; and,
detecting the SARS-CoV-2 proteins when one or more aggregations of the bound SARS-CoV-2 proteins form with one another, thereby detecting the SARS-CoV-2 in the sample.

2. The method of claim 1, wherein the antibodies comprise monoclonal antibodies.

3. The method of claim 1, wherein the first and second set of antibodies, or the antigen binding portions thereof, comprise nanobodies.

4. The method of claim 1, wherein the SARS-CoV-2 protein comprises a receptor-binding domain (RBD).

5. The method of claim 1, wherein the detection step comprises determining a change in absorbance at a resonance wavelength of the AuNPs and/or other MNPs.

6. The method of claim 1, comprising quantifying an amount of the SARS-CoV-2 proteins and/or the SARS-CoV-2 in the sample.

7. The method of claim 1, further comprising centrifuging the aggregations of the bound SARS-CoV-2 proteins prior to and/or during the detecting step.

8. The method of claim 1, further comprising freezing the aggregations of the bound SARS-CoV-2 proteins prior to the detecting step.

9. The method of claim 1, comprising drop casting the aggregations of the bound SARS-CoV-2 proteins prior to the detecting step.

10. The method of claim 1, comprising obtaining the sample from a subject.

11. The method of claim 10, comprising administering one or more therapies to the subject when the SARS-CoV-2 is detected in the sample.

12. The method of claim 10, comprising detecting the SARS-CoV-2 within about 20 minutes or less of obtaining the sample from the subject.

13. The method of claim 10, comprising repeating the method using one or more longitudinal samples obtained from the subject.

14. The method of claim 10, wherein the sample comprises blood, plasma, serum, saliva, sputum, or urine.

15. The method of claim 1, wherein the detecting step comprises measuring a colorimetric change when the one or more aggregations of the bound SARS-CoV-2 proteins form with one another.

16. The method of claim 15, comprising visually detecting the colorimetric change when the one or more aggregations of the bound SARS-CoV-2 proteins form with one another.

17. The method of claim 15, comprising detecting the colorimetric change when the one or more aggregations of the bound SARS-CoV-2 proteins form with one another using a spectrometer.

18. A device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein when the reaction chamber or substrate receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins and one or more aggregations of the bound SARS-CoV-2 proteins to produce a colorimetric change in the reaction chamber; or (ii) conjugated with a single set of antibodies, or antigen binding portions thereof, that bind to an identical epitope from different monomers of a dimerized SARS-CoV-2 protein under conditions sufficient for the antibodies, or the antigen binding portions thereof, to bind to the identical epitope from the different monomers of the dimerized SARS-CoV-2 protein in the sample to produce bound SARS-CoV-2 proteins and one or more aggregations of the bound SARS-CoV-2 proteins to produce a colorimetric change in the reaction chamber.

19. (canceled)

20. (canceled)

21. A system, comprising:
a device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a severe acute respiratory syndrome coronavirus-2 (SARS-CoV-2) protein and wherein at least a second set of antibodies, or antigen binding portions thereof, binds to a second epitope of a SARS-CoV-2 protein when the reaction chamber receives a sample that comprises the SARS-CoV-2 under conditions sufficient for the first and second set of antibodies, or the antigen binding portions thereof, to bind to the first and second epitopes of the SARS-CoV-2 proteins in the sample to produce bound SARS-CoV-2 proteins; or (ii) conjugated with a single set of antibodies, or antigen binding portions thereof, that bind to an identical epitope from different monomers of a dimerized SARS-CoV-2 protein when the reaction chamber receives a sample that comprises the SARS-CoV-2 under conditions sufficient for the antibodies, or the antigen binding portions thereof, to bind to the identical epitope from the different monomers of the dimerized SARS-CoV-2 protein in the sample to produce bound SARS-CoV-2 proteins; and,
an electromagnetic radiation detection apparatus positioned, or positionable, within sufficient proximity to the device to detect one or more colorimetric changes produced in or on the reaction chamber or substrate when one or more aggregations of the bound SARS-CoV-2 proteins form with one another in or on the reaction chamber or substrate.

22. The system of claim 21, wherein the electromagnetic radiation detection apparatus comprises a spectrometer; wherein the electromagnetic radiation detection apparatus comprises a microscope; and/or wherein the electromagnetic radiation detection apparatus comprises a light-emitting diode (LED) that transmits light into and/or through the reaction chamber or substrate and a photodetector that detects light from the reaction chamber or substrate.
